(19) United States
(12) Patent Application Publication
(10) Pub. No.: US 2009/0208500 A1
(43) Pub. Date: Aug. 20, 2009

(54) Method of producing antibodies with improved function

(75) Inventors: John C. Joly, San Mateo, CA (US); Henry B. Lowman, El Granada, CA (US); Domingos Ng, San Francisco, CA (US); Amy Y. Shen, San Mateo, CA (US); Bradley R. Snedecor, Portola Valley, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/447,506

(22) Filed: Jun. 5, 2006

Related U.S. Application Data
(60) Provisional application No. 60/736,982, filed on Nov. 14, 2005.
(60) Provisional application No. 60/687,625, filed on Jun. 3, 2005.

(51) Int. Cl. A61K 39/395; C12P 21/00; C12N 5/10; A61P 35/00

(52) U.S. Cl. 424/134.1; 435/69.6; 435/325

(57) ABSTRACT

The invention provides methods for controlling fucosylation levels and improving ADCC activity in antibodies.

This application claims the benefit of U.S. Provisional Application Ser. Nos. 60/687,625, filed Jun. 3, 2005, and 60/736,982, filed Nov. 14, 2005 the entire disclosures of which are incorporated herein by reference.

### FIELD OF THE INVENTION

The invention relates to the production of antibodies with reduced fucose and improved Fc function.

### BACKGROUND OF THE INVENTION

Recombinant therapeutic proteins are commonly produced in several mammalian host cell lines including murine myeloma NSO and Chinese Hamster Ovary (CHO) cells (Anderson and Krummen, 2002; Chu and Robinson, 2001). Each cell line has advantages and disadvantages in terms of productivity and the characteristics of the proteins produced by the cells. Choices of commercial production cell lines often balance the need for high productivity with the ability to deliver the product quality attributes required of a given product. One important class of therapeutic recombinant proteins which often require high titer processes are monoclonal antibodies. Some monoclonal antibodies need effector functions, mediated through the Fc region, to elicit their biological functions. An example is Rituximab (Rituxan™, Genentech and Biogen-Idec), a chimeric monoclonal antibody which binds to cell surface CD-20 and results in B-cell depletion (Carton et al., 2002; Idusogie et al., 2000). Other antibodies, such as Bevacizumab (Avastin™, Genentech), a humanized anti-VEGF (vascular endothelial growth factor) antibody, do not require Fc effector functions for their activity.

Monoclonal antibodies produced in mammalian host cells contain an N-linked glycosylation site at Asn²⁹⁷ of each heavy chain (two per intact antibody molecule). Glycans on antibodies are typically complex biatennary structures with very low or no bisecting N-acetylglucosamine (bisecting GlcNAc) and high levels of core fucosylation (Saba et al., 2002). Glycan termini contain very low or no terminal sialic acid and variable amounts of galactose. For a review of glycosylation on antibody function, see, e.g., Wright & Morrison Trend Biotechnol. 15:26-31 (1997). Considerable work shows that changes to the sugar composition of the antibody glycan structure can alter Fc effector functions (Kumpel et al., 1994; Kumpel et al., 1995; Schuster et al., 2005; Shields et al., 2002; Umana et al., 1999). The important carbohydrate structures contributing to antibody activity are believed to be the fucose residues attached via α1,6 linkage to the innermost N-acetylglucosamine (GlacNAc) residues of the Fc region N-linked oligosaccharides (Shields et al., 2002; Shinkawa et al. J. Biol. Chem. 278(5): 3466-3473 (2003)). FcγR binding requires the presence of oligosaccharides covalently attached at the conserved Asn297 in the Fc region (Wright & Morrison (1997)). Non-fucosylated structures have recently been associated with dramatically increased in vitro Antibody-Dependent Cellular Cytotoxicity (ADCC) activity (Shields et al., 2002; Shinkawa et al., 2003). Several laboratories, including our own, have successfully employed RNA interference (RNAi) or knock-out techniques to engineer CHO cells to either decrease the FUT8 mRNA transcript levels or knock out gene expression entirely (Mori et al., 2004; Yamane-Ohnuki et al., 2004). Mori et al. 2004 describe converting an established stable antibody producing cell line to one that produces improved ADCC antibodies by engineering the cells to constitutively express siRNA against the FUT8 gene and applying LCA selection. Mori demonstrated the production of antibodies that contained up to 70% non-fucosylated glycan. Niwa R. et al. (Cancer Res. 64(6):2127-2133 (2004)), reported that an anti-CD20 antibody with lower fucose content can prolong the animal survival significantly in the human PBMC-engrafted mouse model.

Historically, antibodies produced in Chinese Hamster Ovary Cells (CHO), one of the most commonly used industrial hosts, contain about 2 to 6% in the population that are nonfucosylated. YB2/0 (rat myeloma) and Lec13 cell line (a lectin mutant of CHO line which has a deficient GDP-mannose 4,6-dehydratase leading to the deficiency of GDP-fucose or GDP-sugar intermediates that are the substrate of α1,6-fucosyltransferase (Ripka et al., 1986)), however, can produce antibodies with 78 to 98% nonfucosylated species. Unfortunately, the yield of antibody from these cells is extremely poor and therefore, these cell lines are not useful to make therapeutic antibody products commercially. The FUT8 gene encodes the α1,6-fucosyltransferase enzyme that catalyzes the transfer of a fucosyl residue from GDP-fucose to position 6 of Asn-linked (N-linked) GlcNac of an N-glycan (Yanagidani et al. 1997. J. Biochem 121:626-632). It is known that the a 1-6 fucosyltransferase is the only enzyme responsible for adding fucose to the N-linked biantennary carbohydrate at Asn297 in the CH2 domain of the IgG antibody.

Antibodies with a mature carbohydrate structure that lacks fucose attached to an Fc region of the antibody are described in US Pat Appl No US 2003/0157108 (Presta, L.). Examples of publications related to “defucosylated” or “fucose-deficient” antibodies including anti-CD20 antibodies include: US 2003/0157108; WO 2000/61739; WO 2001/29246; US 2003/0115614; US 2002/0164328; US 2004/0093621, 2004/0132140, and US 2004/0110704 (all 3 of Kyowa Hakko Kogyo Co., Ltd); US 2004/0110282; US 2004/0109865; WO 2003/085119; WO 2003/084570; WO 2005/035586; WO 2005/035778; WO2005/053742; US 2006/0063254; US 2006/0064781; US 2006/0078990; US 2006/0078991; U.S. Pat. No. 6,602,684 and US 2003/0175884 (Glycart Biotechnology); Yamane-Ohnuki et al. Biotech. Bioeng. 87: 614 (2004); Mori et al. Biotechnology and Bioengineering 88(7): 901-908 (2004); Li et al. (GlycoFi) in Nature Biology online publication 22 Jan. 2006; Niwa R. et al. Cancer Res. 64(6):2127-2133 (2004); Okazaki et al. J. Mol. Biol. 336:1239-1249 (2004); Yamane-Ohnuki et al. Biotech. Bioeng. 87: 614 (2004); Shinkawa et al. J. Biol. Chem. 278(5): 3466-3473 (2003). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)). See also US 2005/0123546 (Umana et al.) on antigen-binding molecules with modified glycosylation.

RNA interference (RNAi) is a highly conserved, sequence-specific posttranscriptional gene silencing mechanism that uses double-stranded RNA (dsRNA) as a signal to trigger the degradation of homologous mRNA. The mediators of sequence-specific mRNA degradation are 21- to 23-nt small interfering RNAs (siRNAs) generated by ribonuclease III cleavage from longer dsRNAs. dsRNA is a potent inducer of type I interferon (IFN) synthesis and is the activator of two classes of IFN-induced enzymes whose products activate the latent ribonuclease RNase L. These nonspecific responses to dsRNA are not triggered by dsRNA shorter than 30 bp. The most predominant processing products are duplexes of 21- and 22-nt RNAs with symmetric 2-nt 3 overhangs, which are also the most efficient mediators of mRNA degradation (Elbashir et al., Nature 411:494-498 (2001; Elbashir et al Methods 26: 199-213 (2002)).

Patents and patent publications concerning CD20 antibodies include U.S. Pat. Nos. 5,776,456, 5,736,137, 5,843,439, 6,399,061, and 6,682,734, as well as US patent application nos. US 2002/0197255A1, US 2003/0021781A1, US 2003/0082172 A1, US 2003/0095963 A1, US 2003/0147885 A1 (Anderson et al.); U.S. Pat. No. 6,455,043B1 and WO00/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); US2001/0018041A1, US2003/0180292A1, WO01/34194 (Hanna and Hariharan); US appln no. US2002/0006404 and WO02/04021 (Hanna and Hariharan); US appln no. US2002/0012665 A1 and WO01/74388 (Hanna, N.); US appln no. US 2002/0058029 A1 (Hanna, N.); US appln no. US 2003/0103971 A1 (Hariharan and Hanna); US appln no. US2002/0009444A1, and WO01/80884 (Grillo-Lopez, A.); WO01/97858 (White, C.); US appln no. US2002/0128488A1 and WO02/34790 (Reff, M.); WO02/060955 (Braslawsky et al.); WO2/096948 (Braslawsky et al.); WO02/079255 (Reff and Davies); U.S. Pat. No. 6,171,586B1, and WO98/56418 (Lam et al.); WO98/58964 (Raju, S.); WO99/22764 (Raju, S.); WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); US appln no. US 2002/0004587A1 and WO01/77342 (Miller and Presta); US appln no. US2002/0197256 (Grewal, I.); US Appln no. US 2003/0157108 A1 (Presta, L.); U.S. Pat. Nos. 6,565,827B1, 6,090,365B1, 6,287,537B1, 6,015,542, 5,843,398, and 5,595,721, (Kaminski et al.); U.S. Pat. Nos. 5,500,362, 5,677,180, 5,721,108, 6,120,767, 6,652,852B1 (Robinson et al.); U.S. Pat. No. 6,410,391B1 (Raubitschek et al.); U.S. Pat. No. 6,224,866B1 and WO00/20864 (Barbera-Guillem, E.); WO01/13945 (Barbera-Guillem, E.); WO00/67795 (Goldenberg); US Appl No. US 2003/0133930 A1 and WO00/74718 (Goldenberg and Hansen); WO00/76542 (Golay et al.); WO01/72333 (Wolin and Rosenblatt); U.S. Pat. No. 6,368,596B1 (Ghetie et al.); U.S. Pat. No. 6,306,393 and US Appln no. US2002/0041847 A1, (Goldenberg, D.); US Appln no. US2003/0026801A1 (Weiner and Hartmann); WO02/102312 (Engleman, E.); US Patent Application No. 2003/0068664 (Albitar et al.); WO03/002607 (Leung, S.); WO 03/049694, US2002/0009427A1, and US 2003/0185796 A1 (Wolin et al.); WO03/061694 (Sing and Siegall); US 2003/0219818 A1 (Bohen et al.); US 2003/0219433 A1 and WO 03/068821 (Hansen et al.); US2002/0136719A1 (Shenoy et al.); WO2004/032828 (Wahl et al.); WO2004/035607 (Teeling et al.); US2004/0093621 (Shitara et al.). See also U.S. Pat. No. 5,849,898 and EP appln no. 330,191 (Seed et al.); U.S. Pat. No. 4,861,579 and EP332,865A2 (Meyer and Weiss); WO95/03770 (Bhat et al.), US 2001/0056066 (Bugelski et al.); WO 2004/035607 (Teeling et al.); WO 2004/056312 (Lowman et al.); US 2004/0093621 (Shitara et al.); and WO 2004/103404 (Watkins et al.). Publications concerning CD20 antibody include: Teeling, J. et al “Characterisation of new human CD20 monoclonal antibodies with potent cytolytic activity against non-Hodgkin's lymphomas” Blood, June 2004; 10.1182.

In the FUT8 knockout cell line as described in Yamane-Ohnuki 2004 and in the Kyowa Hakko patents, antibody production requires transfection of the genes encoding the desired antibody into that established knockout cell line. There is a need for an efficient method of producing antibodies in a desired cell line while controlling the fucose content of the recombinantly engineered antibodies without undergoing the laborious process of creating a FUT8 gene knockout in a selected cell line each time. The present invention satisfies this need and provides other advantages that will be apparent in the detailed descriptions below.

### SUMMARY OF THE INVENTION

One way to improve the binding affinity of an antibody to FcγRIII is to change the amino acid sequence(s) in the Fc region (see Shields et al (2002)). The humanized anti-CD20 antibody variants shown in Table 3 incorporate amino acid substitutions in the Fc that enhance FcγRIII binding and ADCC. The present invention provides a method of producing antibodies with lower fucose content that when combined with the FcγRIII binding enhancing amino acid changes in the Fc region show an additive effect on the affinity for FcγIII and ADCC.

The present invention provides a method of producing an antibody comprising an IgG Fc in a mammalian host cell while reducing the fucose content of the antibody, comprising introducing simultaneously into the host cell, at least one nucleic acid encoding an antibody and a second nucleic acid encoding at least two siRNAs targeting different coding regions of the FUT8 gene sequence of SEQ ID NO. 1, wherein the siRNAs inhibit the expression of FUT8 and reduce the fucosylation level of the antibody.

The present invention also provides a more efficient method of generating an antibody production cell line with simultaneous fucosylation knockdown that produces antibodies with improved ADCC as compared to antibodies synthesized with normal levels of fucosylation in the mammalian cell. Such an approach can be taken to construct cell lines that exhibit high antibody productivity and controlled levels of fucosylation. Such a cell line is useful for scale-up production of antibodies as in commercial production of therapeutic antibodies. Thus, a method is provided for producing an IgG antibody with improved ADCC, comprising introducing simultaneously into the host cell, at least one nucleic acid encoding an antibody and a second nucleic acid encoding at least two siRNAs targeting different coding regions of the FUT8 gene sequence of SEQ ID NO. 1, wherein the antibody and the siRNAs are expressed in the cell to produce an antibody with reduced fucosylation and increased ADCC activity as compared to the antibody produced in the cell in the absence of the siRNAs.

In one embodiment of the method for producing an IgG antibody with improved ADCC, the antibody comprises at least one amino acid alteration in the Fc region that improves antibody binding to FcγRIII and/or ADCC. The antibody can comprise the Fc amino acid substitutions of S298A, E333A, K334A.

The invention also provides methods of making compositions of humanized CD20 binding antibodies lacking fucose.

In one embodiment of the methods of the present invention, the nucleic acid encoding an antibody encodes both a light (L) chain and a heavy (H) chain of the antibody. In one embodiment, the antibody H and L chains and the siRNAs are encoded on the same expression vector. In an alternative embodiment, the H and L chains are encoded on separate expression vectors and in addition, each of the expression vectors encoding the H and L chain also comprises a nucleic acid encoding at least two siRNAs.

In one embodiment of all the preceding methods, the two siRNAs are expressed under the control of separate promoters. Where Pol III promoters are used to drive transcription of the siRNAs in the expression vectors, one siRNA can be expressed under the H1 promoter while the second siRNAi is expressed under a different Pol III promoter, U6.

In a specific embodiment, the first and second siRNA target nucleotide positions 733-751 and 1056-1074, respectively, of the FUT8 gene sequence of SEQ ID NO. 1.

In any of the embodiments of the above methods, preferably the antibody fucosylation level is reduced by at least 90%, more preferably by at least 95%, even more preferably by at least 99%.

Antibodies produced by the above methods are provided.

In a preferred embodiment of all the preceding methods, the antibody is a therapeutic antibody. In one embodiment, the antibody binds CD20. In one embodiment, the antibody binds BR3. In preferred embodiments, the antibody binds CD20 on humans and other primates. In one embodiment the CD20 binding antibody is a humanized antibody. In preferred embodiments the humanized antibody is a humanized 2H7 antibody, preferably one as described in Tables 3 & 4 below. In separate embodiments the humanized antibody comprises one of these pairs of VL and VH regions: the L chain variable region sequence of SEQ ID NO.2 and the H chain variable region sequence of SEQ ID NO.8; L chain variable region sequence of SEQ ID NO.25 and the H chain variable region sequence of SEQ ID NO.22; or L chain variable region sequence of SEQ ID NO.25 and the H chain variable region sequence of SEQ ID NO.33. In specific embodiments the humanized 2H7 antibody comprises L and H chain pairs of SEQ ID NO. 13 and SEQ ID NO. 14; SEQ ID NO. 26 and SEQ ID NO. 27; and SEQ ID NO. 26 and SEQ ID NO. 34.

Other embodiments of humanized anti-CD20 antibodies are hA20 (also known as IMMU-106, or 90Y-hLL2; US 2003/0219433, Immunomedics); and AME-133 (US 2005/0025764; Applied Molecular Evolution/Eli Lilly). In a different embodiment, the CD20 binding antibody is a human antibody, preferably HUMAX-CD20™ (GenMab). In yet a separate embodiment, the CD20 binding antibody is a chimeric antibody, preferred embodiments being rituximab (Genentech, Inc.) and the chimeric cA20 antibody (described in US 2003/0219433, Immunomedics).

In yet another embodiment, the antibody produced by the methods of the present invention is an antibody that binds BR3.

The invention additionally provides a nucleic acid comprising the sequence of SEQ ID NO. 42 and SEQ ID NO. 43 that encodes two siRNA complementary to two different coding regions of the FUT8 gene.

A composition is provided comprising humanized CD20 binding antibodies having an Fc region, and a carrier, wherein at least 95% of the antibodies in the composition lack fucose.

In a preferred embodiment, the host cell is a Chinese Hamster Ovary (CHO) cell or a derivative thereof.

Another aspect is a host cell comprising at least one nucleic acid encoding an antibody and a second nucleic acid encoding at least two siRNAs targeting different coding regions of the FUT8 gene sequence of SEQ ID NO. 1, wherein the host cell expresses the antibody and the siRNAs.

Uses of the preceding fucose deficient antibody compositions for treatment of diseases are also provided.

### BRIEF DESCRIPTION OF THE FIGURES

FIG. 1: An Asn-linked (N-linked) GlcNac of an N-glycan with a fucosyl residue attached.

FIG. 2: Schematic of RNAi technology in mediating inhibition of gene expression.

FIG. 3: Diagram of pSilencer3.1-H1 Puro plasmid used to generate FUT8 specific siRNA. See Example 1.

FIG. 4: RNAi probe sequences. Five sequences were designed according to rules published in the literature. The bold sequences are complimentary to each other and denote the hairpin portion of the RNA produced. Probes 1-5 correspond to RNAi 1-5 in FIG. 5B. See Example 1.

FIGS. 5A and 5B: FIG. 5A shows a schematic of the full length and flag-FUT8 fusion constructs and the probe regions. FIG. 5B shows an immunoblot using the M2 anti-flag antibody to detect flag-tagged partial CHO FUT8 protein. See Example 1.

FIG. 6: Fucose content (as % nonfucosylation) of 2H7 antibodies from RNAi 2 and RNAi 4 transiently transfected cells, as described in Example 2.

FIG. 7A-E: Binding activities of lower fucose containing 2H7 antibodies to different Fcγ receptors: FcγRI (FIG. 7A); FcγRIIA (FIG. 7B); FcγRIIB (FIG. 7C); FcγRIII F158 (FIG. 7D); and FcγRIII V158 (FIG. 7E), as described in Example 2.

FIG. 8. Northern blot analysis. The FUT8 mRNA is at about 3.5 kb similar in size to rat FUT8. Lane 2 and lane 3 show less FUT8 than control in lane 1. See Example 2.

FIGS. 9A and 9B. Flow chart outlining the process for development of clones with less-fucosylation. FIG. 9A: standard cell line development procedure. FIG. 9B: new cell line development procedure with RNAi unit (s) included in expression plasmid.

FIGS. 10A, 10B, and 10C. Configuration of plasmids. FIG. 10A: Control plasmid set with antibody HC and LC on separate plasmids; FIG. 10B: Test plasmids with HC and LC on separate plasmids containing one or two RNAi transcription units; FIG. 10C: Test plasmids with HC and LC on the same plasmids containing one or two RNAi transcription units. Abbreviations: HC, heavy chain; LC, light chain; CMV; cytomegalovirus promoter and enhancer sequence; PUR-DHFR, puromycin and dihydrofolate reductase fusion gene. See Example 4.

FIGS. 11A and 11B. Antibody expression levels of clones from stable transfection, as described in Example 4. For each plasmid transfection, 72 MTX resistant clones were picked and screened by ELISA for antibody expression. FIG. 11A: Expression titers from the CMV.PD.v511.RNAi4 plasmid transfection. FIG. 11B: Expression titers from the CMV.PD.v511.RNAi2.4 plasmid transfection.

FIG. 12. Taqman Analysis of FUT8 mRNA level. Total RNA was purified from clones derived from the CMV.PD.v511.RNAi4 and the CMV.PD.v511.RNAi2.4 plasmid transfections. FUT8 mRNA levels were measured using Taqman primers and probes specific to the FUT8 gene. See Example 4.

FIG. 13. Equal seeding density assay. Two control clones from the CMV.PD.v511 plasmid transfection, two clones from the CMV.PD.v511.RNAi4 plasmid transfection with lowest non-fucosylation, and 4 clones from the CMV.PD.v511.RNAi2.4 plasmid transfection with lowest non-fucosylation were seeded at 5×10⁴ cells/well in a 96-well plate for antibody production. The antibody titers were determined by ELISA. See Example 4.

FIG. 14. Nonfucosylation levels of the humanized 2H7.v511 antibodies produced by clones transfected with RNAi 4 or RNAi2.4 plasmids. 2H7.v511 (v511 in the figure) with about 5% nonfucosylation is included in the assay as a control. See Example 4.

FIGS. 15A and 15B. FcγRIII binding affinities of fucosylation variants of humanized 2H7.v511 antibody. FIG. 15A compares the binding affinity of the antibodies to the F158 low affinity isotype of FcγRIII receptor; FIG. 15B compares the binding affinity to the V158 high affinity receptor isotype. The control was h2H7.v511 with about 5% nonfucosylation. See Example 4.

FIGS. 16A and 16B. ADCC activity assay. Two variants of humanized 2H7, named v16 and v511 as well as their non-fucosylation (NF) variants were compared for ADCC activity in a cell based assay using Wil2-S cells. 2h7.v16 and .v511 antibody compositions have about 5% nonfucosylation. V16-NF and v511-NF variants have about 65-70% nonfucosylation. FIG. 16A shows the ADCC activity using VF158 donor NK cells in the assay and FIG. 16B shows the activity using VV158 donor cells.

FIG. 17 shows the DNA sequence (SEQ ID NO. 1) encoding the full length CHO FUT8.

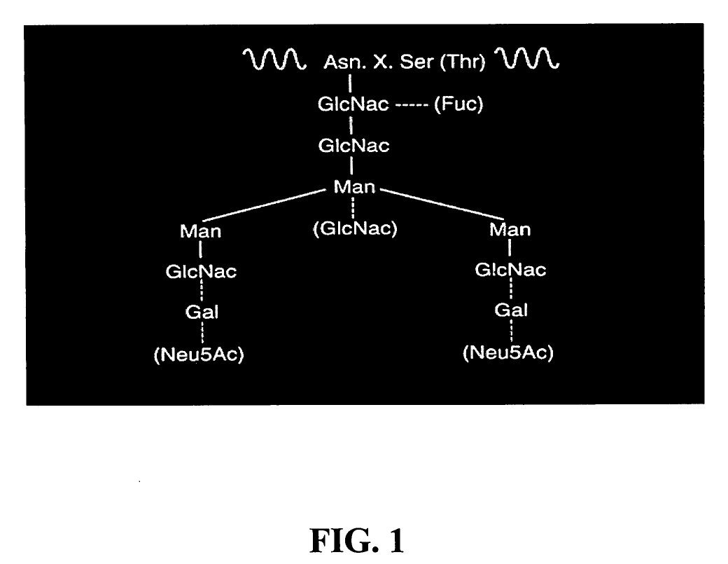

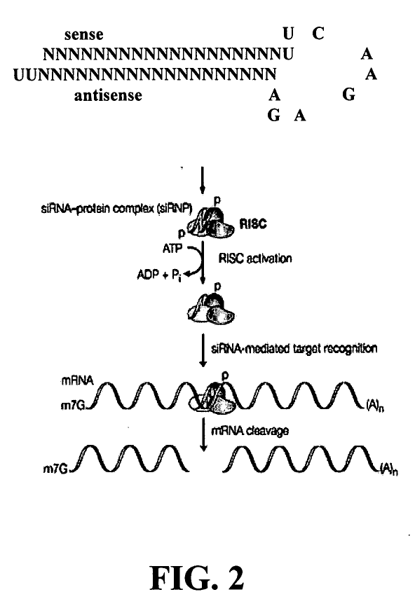

### DETAILED DESCRIPTION OF THE PREFERRED EMBODIMENTS

The “CD20” antigen is a non-glycosylated, transmembrane phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells or normal plasma cells. CD20 is present on both normal B cells as well as malignant B cells. Other names for CD20 in the literature include “B-lymphocyte-restricted differentiation antigen” and “Bp35”. The CD20 antigen is described in, for example, Clark and Ledbetter, Adv. Can. Res. 52:81-149 (1989) and Valentine et al. J. Biol. Chem. 264(19):11282-11287 (1989).

The term “antibody” is used in the broadest sense and specifically covers monoclonal antibodies (including full length monoclonal antibodies), multispecific antibodies (e.g., bispecific antibodies), and antibody fragments so long as they exhibit the desired biological activity or function.

The biological activity of the humanized CD20 binding antibodies of the invention will include at least binding of the antibody to human CD20, more preferably binding to human and other primate CD20 (including cynomolgus monkey, rhesus monkey, chimpanzees, baboons). The antibodies would bind CD20 with a K_{d} value of no higher than 1×10⁻⁸, preferably a K_{d} value no higher than about 1×10⁻⁹, and be able to kill or deplete B cells in vivo, preferably by at least 20% when compared to the appropriate negative control which is not treated with such an antibody. B cell depletion can be a result of one or more of ADCC, CDC, apoptosis, or other mechanism. In some embodiments of disease treatment herein, specific effector functions or mechanisms may be desired over others and certain variants of the humanized 2H7 are preferred to achieve those biological functions, such as ADCC.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody from a population of substantially homogeneous antibodies, i.e., the individual antibodies comprising the population are identical in primary amino acid sequence and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); 5,545,807; WO 1997/17852; U.S. Pat. Nos. 5,545,807; 5,545,806; 5,569,825; 5,625,126; 5,633,425; and 5,661,016; Marks et al., BioTechnology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

“Functional fragments” of the CD20 binding antibodies of the invention are those fragments that retain binding to CD20 with substantially the same affinity as the intact full length molecule from which they are derived and show biological activity including depleting B cells as measured by in vitro or in vivo assays such as those described herein.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As referred to herein, the “consensus sequence” or consensus V domain sequence is an artificial sequence derived from a comparison of the amino acid sequences of known human immunoglobulin variable region sequences. Based on these comparisons, recombinant nucleic acid sequences encoding the V domain amino acids that are a consensus of the sequences derived from the human κ and the human H chain subgroup III V domains were prepared. The consensus V sequence does not have any known antibody binding specificity or affinity.

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Humanized antibody as used herein is a subset of chimeric antibodies.

“Humanized” forms of non-human (e.g., murine) antibodies are chimeric antibodies which contain minimal sequence derived from non-human immunoglobulin. For the most part, humanized antibodies are human immunoglobulins (recipient or acceptor antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or Presta U.S. Pat. No. 6,737,056 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). Where the antibody is a CD20 binding antibody, ADCC activity can be tested in transgenic mice expressing human CD20 plus CD16 (hCD20+/hCD16+ Tg mice) as described below.

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g. from blood.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses; including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulates homeostasis of immunoglobulins.

WO00/42072 (Presta) describes antibody variants with improved or diminished binding to FcRs. The content of that patent publication is specifically incorporated herein by reference. See, also, Shields et al. J. Biol. Chem. 9(2): 6591-6604 (2001).

For binding affinity to FcRn, in one embodiment, the EC50 or apparent Kd (at pH 6.0) of the antibody is <=100 nM, more preferably <=10 nM. For increased binding affinity to FcγRIII (F158; i.e. low-affinity isotype), in one embodiment the EC50 or apparent Kd <=10 nM, and for FcgRIII (V158; high-affinity) the EC50 or apparent Kd <=3 nM. Methods of measuring binding to FcRn are known (see, e.g., Ghetie 1997, Hinton 2004) as well as described below. Binding to human FcRn in vivo and serum half life of human FcRn high affinity binding polypeptides can be assayed, e.g, in transgenic mice or transfected human cell lines expressing human FcRn, or in primates administered with the Fc variant polypeptides. In certain embodiments, the humanized 2H7 antibody of the invention further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed.

Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

Throughout the present specification and claims, unless otherwise indicated, the numbering of the residues in the constant domains of an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody. The residues in the V region are numbered according to Kabat numbering unless sequential or other numbering system is specifically indicated.

Examples of CD20 antibodies include: “C2B8,” which is now called “rituximab” (“RITUXAN®/MABTHERA®”) (U.S. Pat. No. 5,736,137); the yttrium-[90]-labelled 2B8 murine antibody designated “Y2B8” or “Ibritumomab Tiuxetan” (ZEVALIN®) commercially available from Biogen Idec, Inc. (e.g., U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™) commercially available from Corixa (see, also, e.g., U.S. Pat. No. 5,595,721); murine monoclonal antibody “1F5” (e.g., Press et al. Blood 69(2):584-591 (1987) and variants thereof including “framework patched” or humanized 1F5 (e.g., WO 2003/002607, Leung, S.; ATCC deposit HB-96450); murine 2H7 and chimeric 2H7 antibody (e.g., U.S. Pat. No. 5,677,180); a humanized 2H7 (e.g., WO 2004/056312 (Lowman et al.) and as set forth below); HUMAX-CD20™ fully human, high-affinity antibody targeted at the CD20 molecule in the cell membrane of B-cells (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO 2004/035607 and WO 2005/103081 (Teeling et al., GenMab/Medarex); the antibodies having complex N-glycoside-linked sugar chains bound to the Fc region described in US 2004/0093621 (Shitara et al.); monoclonal antibodies and antigen-binding fragments binding to CD20 (e.g., WO 2005/000901, Tedder et al.) such as HB20-3, HB20-4, HB20-25, and MB20-11; single-chain proteins binding to CD20 (e.g., US 2005/0186216 (Ledbetter and Hayden-Ledbetter); US 2005/0202534 (Hayden-Ledbetter and Ledbetter); US 2005/0202028 (Hayden-Ledbetter and Ledbetter); US 2005/0202023 (Hayden-Ledbetter and Ledbetter)—Trubion Pharm Inc.); CD20-binding molecules such as the AME series of antibodies, e.g., AME-133™ antibodies as set forth, for example, in WO 2004/103404 and US 2005/0025764 (Watkins et al., Applied Molecular Evolution, Inc.) and the CD20 antibodies with Fc mutations as set forth, for example, in WO 2005/070963 (Allan et al., Applied Molecular Evolution, Inc.); CD20-binding molecules such as those described in WO 2005/016969 and US 2005/0069545 (Carr et al.); bispecific antibodies as set forth, for example, in WO 2005/014618 (Chang et al.); humanized LL2 monoclonal antibodies as described, for example, in US 2005/0106108 (Leung and Hansen; Immunomedics); chimeric or humanized B-Ly1 antibodies to CD20 as described, for example, in WO2005/044859 and US 2005/0123546 (Umana et al.; GlycArt Biotechnology AG); A20 antibody or variants thereof such as chimeric or humanized A20 antibody (cA20, hA20, respectively) and IMMUN-106 (e.g., US 2003/0219433, Immunomedics); and monoclonal antibodies L27, G28-2, 93-1B3, B-C1 or NU-B2 available from the International Leukocyte Typing Workshop (e.g., Valentine et al., In: Leukocyte Typing III (McMichael, Ed., p. 440, Oxford University Press (1987)). The preferred CD20 antibodies herein are chimeric, humanized, or human CD20 antibodies, more preferably rituximab, a humanized 2H7, chimeric or humanized A20 antibody (Immunomedics), HUMAX-CD20™ human CD20 antibody (Genmab), and immunoglobulins/proteins binding to CD20 (Trubion Pharm Inc.).

The terms “BR3”, “BR3 polypeptide” or “BR3 receptor” when used herein encompass “native sequence BR3 polypeptides”. Human BR3 sequence (SEQ ID NO: 44)

| | |
|---|---|
| 1 | MRRGPRSLRG RDAPAPTPCV PAECFDLLVR HCVACGLLRT PRPKPAGASS PAPRTALQPQ |
| 61 | ESVGAGAGEA ALPLPGLLFG APALLGLALV LALVLVGLVS WRRRQRRLRG ASSAEAPDGD |
| 121 | KDAPEPLDKV IILSPGISDA TAPAWPPPGE DPGTTPPGHS VPVPATELGS TELVTTKTAG |
| 181 | PEQQ |

As used herein, “B cell depletion” refers to a reduction in B cell levels in an animal or human after drug or antibody treatment, as compared to the level before treatment. B cell levels are measurable using well known assays such as by getting a complete blood count, by FACS analysis staining for known B cell markers, and by methods such as described in the Experimental Examples. B cell depletion can be partial or complete. In one embodiment, the depletion of CD20 expressing B cells is at least 25%. In a patient receiving a B cell depleting drug, B cells are generally depleted for the duration of time when the drug is circulating in the patient's body and the time for recovery of B cells.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Vector” includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells. Suitable vectors are disclosed below.

The cell that produces a humanized CD20 binding antibody such as humanized 2H7 antibody of the invention will include the bacterial and eukaryotic host cells into which nucleic acid encoding the antibodies have been introduced. Suitable host cells are disclosed below.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

### Methods and Compositions of the Invention

RNAi Interference

Long double-stranded RNAs (dsRNAs; typically >200 nt) can be used to silence the expression of target genes in a variety of organisms and cell types (e.g., worms, fruit flies, and plants). Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase E1-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand leading to specific gene silencing. However, since most mammalian cells mount a potent antiviral response characterized by nonspecific inhibition of protein synthesis and RNA degradation upon introduction of dsRNA longer than 30 bp, researchers transfect cells with 21-23 bp siRNAs to induce RNAi in these systems without eliciting the antiviral response. In the present method of the invention, at least one specific dsRNA that targets a particular gene transcript (FUT8 in this case) is used to induce the RNAi pathway. The dsRNA is delivered into the cell by any suitable dsRNA delivery system. An appropriate negative control would be a dsRNA that does not target any transcript in the organism (e.g., dsRNA targeting luciferase).

In the present method of the invention, at least one specific dsRNA that targets a particular gene transcript (FUT8 in this case) is used to induce the RNAi pathway. In mammalian cultured cells, RNAi is typically induced by siRNA introduced directly or expressed as a hairpin structure from a DNA construct within the cells.

Methods to Produce siRNA

There are 5 commonly known methods for generating siRNAs for gene silencing studies: (i) chemical synthesis; (ii) in vitro transcription; (iii) digestion of long dsRNA by an RNase III family enzyme (e.g. Dicer, RNase III); (iv) expression in cells from an siRNA expression plasmid or viral vector; and (vi) expression in cells from a PCR-derived siRNA expression cassette. The first three methods involve in vitro preparation of siRNAs that are then introduced directly into mammalian cells by lipofection, electroporation, or other technique. The last two methods rely on the introduction of DNA-based vectors and cassettes that express siRNAs within the cells. All of these methods, except creation of siRNA populations by digestion of long dsRNA, require careful design of the siRNA to maximize silencing of the target gene while minimizing the effects on off-target genes. Chemical synthesis is the preferred and most widely used method of siRNA preparation for transient transfection of cultured mammalian cells followed by a downstream assay to monitor the RNAi effect. siRNAs are easier to transfect than plasmids.

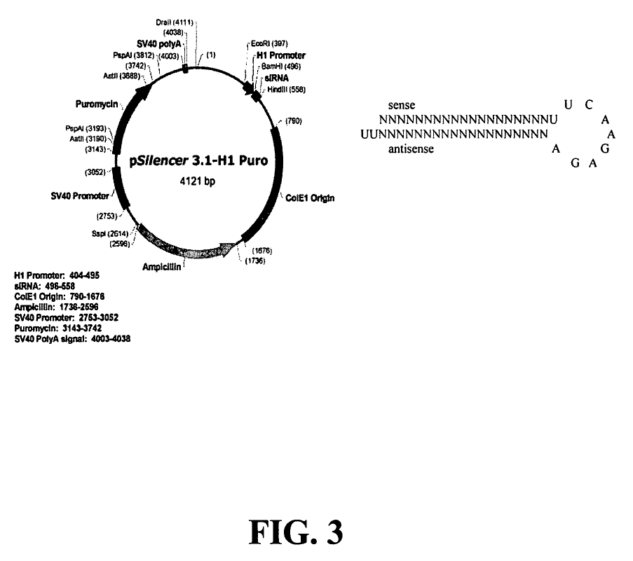

Exemplary siRNA expression vectors are the pSilencer™ siRNA expression vectors from Ambion, Inc., (Austin, Tex.) which express siRNA within mammalian cells using a U6 (Kunkel and Pederson, 1988; Miyashi and Taira, 2002) or H1 Polymerase III promoter. For example, pSilencer 3.0-H1 (plasmid components shown in FIG. 3) features the H1 RNA promoter (H1 RNA is a component of RNase P). Various selectable markers such as hygomcyin, neomycin, puromcyin can be included in these vectors. The pSilencer 2.0-U6 and 3.0-H1 siRNA expression vectors are linearized with BamH I and Hind III, which leave overhangs that facilitate directional cloning. To elicit silencing, a small DNA insert encoding a short hairpin RNA targeting the gene of interest is cloned into the vector downstream of the Pol III promoter. Once transfected into mammalian cells, the insert-containing vector expresses the short hairpin RNA, which is rapidly processed by the cellular machinery into siRNA.

Delivery of siRNAs into Cultured Cells

For many immortalized cell lines, transfection of the siRNA can be performed with a lipid- or amine-based reagent, e.g., Ambion's siPORT™ Lipid and siPORT™ Amine Transfection Agents. For delivery into primary cells and suspension cells, electroporation using a specialized, gentle-on-cells buffer and optimized pulsing conditions generally results in very efficient siRNA delivery without compromising cell viability.

Controls for siRNA Experiments

A negative control that does not target any endogenous transcript (e.g., dsRNA targeting luciferase) is useful to control for nonspecific effects on gene expression caused by introducing any siRNA. Easy-to-assay positive controls are useful to optimize transfection conditions, ensure that siRNAs are efficiently delivered, and ascertain that a particular downstream assay is working. Since positive controls are used for many different aspects of an RNAi experiment, often more than one control is required. For transfection optimization experiments, Silencer™ GAPDH siRNA is an ideal positive control. This siRNA efficiently silences GAPDH expression and its effects can be easily monitored by qRT-PCR or other methods at the mRNA level, or by Western blot or immunofluorescence at the protein level.

Assay for RNAi Effect

There are several assays for measuring the RNAi effect. Assays that can be used for understanding the biological effects of knocking down a target gene include cell based assays, enzymatic assays, array analysis. siRNAs exert their effects at the mRNA level. The simplest assay for siRNA validation and transfection optimization relies on qRT-PCR to measure target transcript levels in gene specific siRNA treated cells versus negative control siRNA treated cells. Applied Biosystems' TaqMan® Gene Expression Assays, available for >41,000 human, mouse, and rat genes, are also useful for this purpose. Ambion's siRNA database provides links to individual assays matched to gene specific Silencer™ Pre-designed and Validated siRNAs. The extent of knockdown at the protein level can also be assessed. Since native protein is recovered in most cases, enzymatic assays can also be performed. siRNA, target mRNA, and target protein levels can be correlated.

The antibodies of the invention comprise IgG Fc regions and normally bind to FcγRIIIA and exhibit ADCC in vitro and in vivo. The mammalian host cell commonly used to produce antibodies having an IgG Fc region or fragment thereof that retain the Asn glycosylation site and ADCC effector function, generally produce a population of antibodies of which 94-98% of the monoclonal antibodies in the population are fucosylated. The transfectant cells generated by the method of the present invention and expressing 2 or more siRNA targeting the FUT8 gene will produce a population of the desired antibody that has reduced fucosylation levels compared to the antibody population produced by host cells that have normal, unmodified FUT8 expression and as a result, the reduced fucosylated population of antibodies as a whole is capable of improved FcγRIIIA and/or ADCC in the presence of the appropriate effector cells.

In one embodiment, the reduced fucosylation antibodies produced by the method of the invention bind CD20, in particular, primate CD20. In one embodiment, these antibodies bind human CD20. In one

In one embodiment, the invention provides humanized 2H7 antibodies having reduced fucose that are generated by the methods of the invention. The generation of hu2H7 antibodies are described in detail in WO 04/056312 incorporated herein by reference in its entirety. In specific embodiments, the variant is 2H7.v16, hu2H7.v511 and hu2H7.v114.

In a full length antibody, the humanized CD20 binding antibody of the invention will comprise a humanized V domain joined to a C domain of a human immunoglobulin. In a preferred embodiment, the H chain C region is from human IgG, preferably IgG1 or IgG3. The L chain C domain is preferably from human κ chain.

For the purposes herein, “humanized 2H7” refers to an intact antibody or antibody fragment comprising the variable light (V_{L}) sequence:

(SEQ ID NO: 2)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKR;

and
variable heavy (V_{H}) sequence:

(SEQ ID NO: 8)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSS

Where the humanized 2H7 antibody is an intact antibody, preferably it comprises the v16 light chain amino acid sequence:

(SEQ ID NO: 13)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC;

and
heavy chain amino acid sequence:

(SEQ ID NO: 14)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G.

A variant of the preceding humanized 2H7 mAb is 2H7v.31 having the same L chain sequence as SEQ ID NO: 13 above, and comprising the H chain amino acid sequence:

(SEQ ID NO: 15)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYTTFP
PVLDSDGSFFLYSKLTVDKSRWQQGNVFSCSVMHEALHNHYTQKSLSLSP
GK.

Another variant of the preceding humanized 2H7 antibody is one that comprises the

V_{L} of SEQ ID NO.25

(SEQ ID NO. 25)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR;

and

the VH of SEQ ID NO. 22 of 2H7.v114:

(SEQ ID NO. 22)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSS

The complete L chain amino acid sequence of 2H7v.114 has the following sequence

(SEQ ID NO: 26)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

The complete H chain amino acid sequence of 2H7v.114:

(SEQ ID NO:27)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK

Yet another variant is 2H7.v138 comprising the H chain amino acid sequence of SEQ ID NO. 26.
An additional variant, 2H7.v477, comprises the V_{L} of SEQ ID NO. 25 and the VH of SEQ ID NO. 22 and has the H chain amino acid sequence:

(SEQ ID NO: 31)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHWHYTQKSLSLSP
GK.

Yet another variant of the preceding humanized 2H7 antibody is one that comprises the V_{L} of SEQ ID NO. 25 and VH of SEQ ID NO. 33 of 2H7.v511 [see Table 4]

(SEQ ID NO. 33)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSS

In one embodiment the antibody comprises the 2H7.v511 Light Chain (SEQ ID NO.26)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTYHQG
LSSPVTKSFNRGEC

And the 2H7.v511 Heavy Chain (SEQ ID NO. 34)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G

The V region of all other variants based on version 16 will have the amino acid sequences of v16 except at the positions of amino acid substitutions which are indicated in Table 3 below. Unless otherwise indicated, the 2H7 variants will have the same L chain as that of v16. Humanized antibody 2H7v.16 is also referred to as rhuMAb2H7, PRO70769, or Ocrelizumab.

TABLE 3

| | Light chain | Heavy chain | |
|---|---|---|---|
| 2H7 version | (V_{L}) changes | (V_{H}) changes | Fc changes |
| 16 for reference | | | — |
| 31 | — | — | S298A, E333A, K334A |
| 73 | M32L | N100A | |
| 75 | M32L | N100A | S298A, E333A, K334A |
| 96 | S92A | D56A, N100A | |
| 114 | M32L, S92A | D56A, N100A | S298A, E333A, K334A |
| 115 | M32L, S92A | D56A, N100A | S298A, E333A, K334A, E356D, M358L |
| 116 | M32L, S92A | D56A, N100A | S298A, K334A, K322A |
| 138 | M32L, S92A | D56A, N100A | S298A, E333A, K334A, K326A |
| 477 | M32L, S92A | D56A, N100A | S298A, E333A, K334A, K326A, N434W |
| 375 | — | — | K334L |
| 511 | M32L, S92A | D56A, N100Y, | S298A, E333A, K334A, K326A, |
| | | S100aR | |
| 588 | — | — | S298A, E333A, K334A, K326A |

TABLE 4

| | V_{L} | V_{H} | Full L chain | Full H chain |
|---|---|---|---|---|
| 2H7 version | SEQ ID NO. | SEQ ID NO. | SEQ ID NO. | SEQ ID NO. |
| 16 | 2 | 8 | 13 | 14 |
| 31 | 2 | 8 | 13 | 15 |
| 73 | 16 | 17 | 18 | 19 |
| 75 | 16 | 17 | 18 | 20 |
| 96 | 21 | 22 | 23 | 24 |
| 114 | 25 | 22 | 26 | 27 |
| 115 | 25 | 22 | 26 | 28 |
| 116 | 25 | 22 | 26 | 29 |
| 138 | 25 | 22 | 26 | 30 |
| 477 | 25 | 22 | 26 | 31 |
| 375 | 2 | 8 | 13 | 32 |
| 511 | 25 | 33 | 26 | 34 |
| 588 | 2 | 8 | 35 | 36 |

Residue numbering is according to Kabat et al., Sequences of Immunological Interest. 5th Ed. Public Health Service, National Institutes of Health, Bethesda, Md. (1991), with insertions shown as a, b, c, d, and e, and gaps shown as dashes in the sequence figures. In the CD20 binding antibodies that comprise Fc region, the C-terminal lysine (residue 447 according to the EU numbering system) of the Fc region may be removed, for example, during purification of the Ab or by recombinant engineering the nucleic acid encoding the antibody polypeptide. Accordingly, a humanized 2H7 antibody composition of this invention can comprise antibody with K447, with all K447 removed, or a mixture of antibody with and without the K447 residue.

The N-glycosylation site in IgG is at Asn297 in the CH2 domain. Humanized 2H7 antibody compositions of the present invention include compositions of any of the preceding humanized 2H7 antibodies having a Fc region, wherein about 80-100% (and preferably about 90-99%) of the antibody in the composition comprises a mature core carbohydrate structure which lacks fucose, attached to the Fc region of the glycoprotein. Such compositions were demonstrated herein to exhibit a surprising improvement in binding to FcγRIIIA (F158), which is not as effective as FcγRIIIA (V158) in interacting with human IgG. FcγRIIIA (F158) is more common than FcγRIIIA (V158) in normal, healthy African Americans and Caucasians. See Lehrnbecher et al. Blood 94:4220 (1999).

A bispecific humanized 2H7 antibody encompasses an antibody wherein one arm of the antibody has at least the antigen binding region of the H and/or L chain of a humanized 2H7 antibody of the invention, and the other arm has V region binding specificity for a second antigen. In specific embodiments, the second antigen is selected from the group consisting of CD3, CD64, CD32A, CD16, NKG2D or other NK activating ligands.

In certain embodiments, the humanized 2H7 antibody of the invention further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

Expression of FUT8 is inhibited or knocked down if the level of FUT8 transcripts or protein in the siRNA transfected cell is measurably reduced as compared to the level in the same without transfection and expression of the FUT8 inhibitory siRNA. FUT8 transcripts or protein in the cell and the fucose content of the antibodies produced can be quantitated by the methods described below. Preferably the level of inhibition of FUT8 expression results in a reduction in the fucosylation level of the antibodies in the composition by at least 65%, preferably by 75-80%, more preferably by 90%, even more preferably by 95% or 99%.

Promoters useful to drive the siRNA expression are Pol III type promoters such as H1 or U6 promoter. tRNA promoters can also be used.

Host cells will include eukaryotic cells such as mammalian and plants cells. Preferably the host cell is a mammalian cell such as CHO cell but other suitable host cells are provided herein.

FcγRIII binding and/or ADCC is improved if the antibody exhibits a level of binding and ADCC activity increased over that from the same antibody produced in the host cell with normal FUT8 gene function without RNAi and FUT8 knockdown. Methods of measuring FcγR binding and ADCC are described below.

### Antibody Production

Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., BioTechnology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The humanized antibody may be an antibody fragment, such as a Fab, which is optionally conjugated with one or more cytotoxic agent(s) in order to generate an immunoconjugate. Alternatively, the humanized antibody may be an full length antibody, such as an full length IgG1 antibody.

Human Antibodies and Phage Display Methodology

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

Bispecific Antibodies

Bispecific antibodies are antibodies that have binding specificities for at least two different epitopes. Exemplary bispecific antibodies may bind to two different epitopes of the CD20 protein. Other such antibodies may combine a CD20 binding site with a binding site for another protein. Alternatively, an anti-CD20 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), or NKG2D or other NK cell activating ligand, so as to focus and localize cellular defense mechanisms to the CD20-expressing cell. Bispecific antibodies may also be used to localize cytotoxic agents to cells which express CD20. These antibodies possess a CD20-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Other Amino Acid Sequence Modifications

Amino acid sequence modification(s) of the CD20 binding antibodies described herein are contemplated. For example, it may be desirable to improve the binding affinity and/or other biological properties of the antibody. Amino acid sequence variants of the anti-CD20 antibody are prepared by introducing appropriate nucleotide changes into the anti-CD20 antibody nucleic acid, or by peptide synthesis. Such modifications include, for example, deletions from, and/or insertions into and/or substitutions of, residues within the amino acid sequences of the anti-CD20 antibody. Any combination of deletion, insertion, and substitution is made to arrive at the final construct, provided that the final construct possesses the desired characteristics. The amino acid changes also may alter post-translational processes of the anti-CD20 antibody, such as changing the number or position of glycosylation sites.

A useful method for identification of certain residues or regions of the anti-CD20 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with CD20 antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-CD20 antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-CD20 antibody with an N-terminal methionyl residue or the antibody fused to a cytotoxic polypeptide. Other insertional variants of the anti-CD20 antibody molecule include the fusion to the N- or C-terminus of the anti-CD20 antibody to an enzyme (e.g. for ADEPT) or a polypeptide which increases the serum half-life of the antibody.

Another type of variant is an amino acid substitution variant. These variants have at least one amino acid residue in the anti-CD20 antibody molecule replaced by a different residue. The sites of greatest interest for substitutional mutagenesis include the hypervariable regions, but FR alterations are also contemplated. Conservative substitutions are shown in the Table below under the heading of “preferred substitutions”. If such substitutions result in a change in biological activity, then more substantial changes, denominated “exemplary substitutions” in the Table, or as further described below in reference to amino acid classes, may be introduced and the products screened.

TABLE of Amino Acid Substitutions

| | Exemplary | Preferred |
|---|---|---|
| Original Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the anti-CD20 antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human CD20. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-CD20 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-CD20 antibody.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement mediated lysis and ADCC capabilities. See Stevenson et al. Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Screening for Antibodies with the Desired Properties

Antibodies with certain biological characteristics may be selected as described in the Experimental Examples.

The growth inhibitory effects of an anti-CD20 antibody of the invention may be assessed by methods known in the art, e.g., using cells which express CD20 either endogenously or following transfection with the CD20 gene. For example, tumor cell lines and CD20-transfected cells may treated with an anti-CD20 monoclonal antibody of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an anti-CD20 antibody of the invention. After antibody treatment, the cells are harvested and the amount of radioactivity incorporated into the DNA quantitated in a scintillation counter. Appropriate positive controls include treatment of a selected cell line with a growth inhibitory antibody known to inhibit growth of that cell line.

To select for antibodies which induce cell death, loss of membrane integrity as indicated by, e.g., propidium iodide (PI), trypan blue or 7AAD uptake may be assessed relative to control. A PI uptake assay can be performed in the absence of complement and immune effector cells. CD20-expressing tumor cells are incubated with medium alone or medium containing of the appropriate monoclonal antibody at e.g, about 10 μg/ml. The cells are incubated for a 3 day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN™ flow cytometer and FACSCONVERT™ CellQuest software (Becton Dickinson). Those antibodies which induce statistically significant levels of cell death as determined by PI uptake may be selected as cell death-inducing antibodies.

To screen for antibodies which bind to an epitope on CD20 bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as an anti-CD20 antibody of the invention. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of CD20 can be used in competition assays with the test antibodies or with a test antibody and an antibody with a characterized or known epitope.

### Vectors, Host Cells and Recombinant Methods

The invention also provides an isolated nucleic acid encoding a humanized 2H7 variant antibody, vectors and host cells comprising the nucleic acid, and recombinant techniques for the production of the antibody.

For recombinant production of the antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

(i) Signal Sequence Component

The humanized 2H7 antibody of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process the native CD20 binding antibody signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the humanized 2H7 antibody.

(ii) Origin of Replication

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the nucleic acid encoding the humanized 2H7 antibody, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding the humanized 2H7 antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

(iv) Promoter Component

Expression and cloning vectors usually contain a promoter that is recognized by the host organism and is operably linked to the nucleic acid encoding the humanized 2H7 antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the CD20 binding antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Humanized 2H7 antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of a DNA encoding the humanized 2H7 antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the CD20 binding antibody-encoding sequence, but is preferably located at a site 5′ from the promoter.

(vi) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding CD20 binding antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for CD20 binding antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated humanized 2H7 antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for CD20 binding antibody production and cultured in conventional nutrient media modified as appropriate for inducing promoters, selecting transformants, or amplifying the genes encoding the desired sequences.

(viii) Culturing the Host Cells

The host cells used to produce the CD20 binding antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., BioTechnology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human β3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Antibody Conjugates

The antibody may be conjugated to a cytotoxic agent such as a toxin or a radioactive isotope. In certain embodiments, the toxin is calicheamicin, a maytansinoid, a dolastatin, auristatin E and analogs or derivatives thereof, are preferable.

Preferred drugs/toxins include DNA damaging agents, inhibitors of microtubule polymerization or depolymerization and antimetabolites. Preferred classes of cytotoxic agents include, for example, the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins and differentiation inducers. Particularly useful members of those classes include, for example, methotrexate, methopterin, dichloromethotrexate, 5-fluorouracil, 6-mercaptopurine, cytosine arabinoside, melphalan, leurosine, leurosideine, actinomycin, daunorubicin, doxorubicin, N-(5,5-diacetoxypentyl)doxorubicin, morpholino-doxorubicin, 1-(2-choroehthyl)-1,2-dimethanesulfonyl hydrazide, N⁸-acetyl spermidine, aminopterin methopterin, esperamicin, mitomycin C, mitomycin A, actinomycin, bleomycin, caminomycin, aminopterin, tallysomycin, podophyllotoxin and podophyllotoxin derivatives such as etoposide or etoposide phosphate, vinblastine, vincristine, vindesine, taxol, taxotere, retinoic acid, butyric acid, N⁸-acetyl spermidine, camptothecin, calicheamicin, bryostatins, cephalostatins, ansamitocin, actosin, maytansinoids such as DM-1, maytansine, maytansinol, N-desmethyl-4,5-desepoxymaytansinol, C-19-dechloromaytansinol, C-20-hydroxymaytansinol, C-20-demethoxymaytansinol, C-9-SH maytansinol, C-14-alkoxymethylmaytansinol, C-14-hydroxy or acetyloxymethlmaytansinol, C-15-hydroxy/acetyloxymaytansinol, C-15-methoxymaytansinol, C-18-N-demethylmaytansinol and 4,5-deoxymaytansinol, auristatins such as auristatin E, M, PHE and PE; dolostatins such as dolostatin A, dolostatin B, dolostatin C, dolostatin D, dolostatin E (20-epi and 11-epi), dolostatin G, dolostatin H, dolostatin I, dolostatin 1, dolostatin 2, dolostatin 3, dolostatin 4, dolostatin 5, dolostatin 6, dolostatin 7, dolostatin 8, dolostatin 9, dolostatin 10, deo-dolostatin 10, dolostatin 11, dolostatin 12, dolostatin 13, dolostatin 14, dolostatin 15, dolostatin 16, dolostatin 17, and dolostatin 18; cephalostatins such as cephalostatin 1, cephalostatin 2, cephalostatin 3, cephalostatin 4, cephalostatin 5, cephalostatin 6, cephalostatin 7, 25′-epi-cephalostatin 7, 20-epi-cephalostatin 7, cephalostatin 8, cephalostatin 9, cephalostatin 10, cephalostatin 11, cephalostatin 12, cephalostatin 13, cephalostatin 14, cephalostatin 15, cephalostatin 16, cephalostatin 17, cephalostatin 18, and cephalostatin 19.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al. Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene.

There are many linking groups known in the art for making antibody-maytansinoid conjugates, including, for example, those disclosed in U.S. Pat. No. 5,208,020 or EP Patent 0 425 235 B1, and Chari et al. Cancer Research 52: 127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Calicheamicin

Another immunoconjugate of interest comprises an CD20 binding antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ^{I}₁ (Hinman et al. Cancer Research 53: 3336-3342 (1993), Lode et al. Cancer Research 58: 2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Radioactive Isotopes

For selective destruction of the tumor, the antibody may comprise a highly radioactive atom. A variety of radioactive isotopes are available for the production of radioconjugated anti-CD20 antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al. Cancer Research 52: 127-131 (1992); U.S. Pat. No. 5,208,020) may be used.

### Therapeutic Uses

The humanized 2H7 CD20 binding antibodies of the invention are useful to treat a number of malignant and non-malignant diseases including CD20 positive cancers such as B cell lymphomas and leukemia, and autoimmune diseases. Stem cells (B-cell progenitors) in bone marrow lack the CD20 antigen, allowing healthy B-cells to regenerate after treatment and return to normal levels within several months. hu2H7.v511 is the preferred antibody to be used in the treatment methods herein.

CD20 positive cancers are those comprising abnormal proliferation of cells that express CD20 on the cell surface. The CD20 positive B cell neoplasms include CD20-positive Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia.

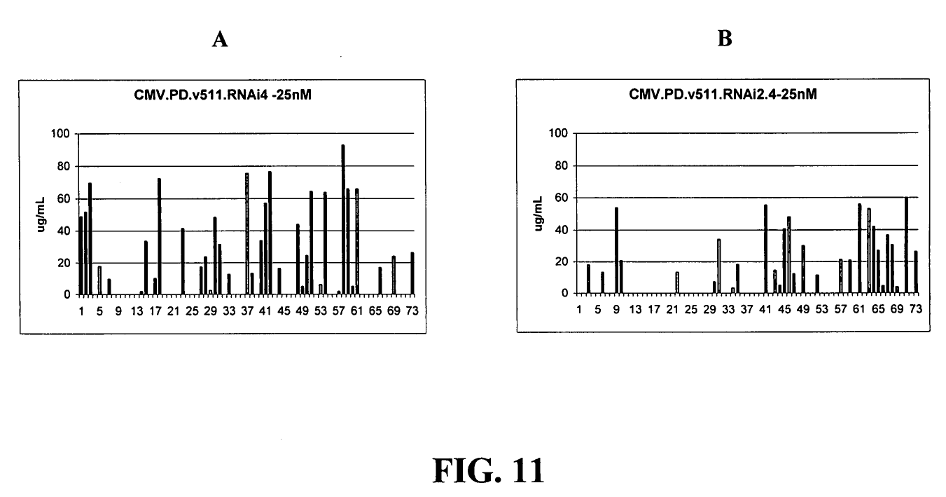

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Color Atlas of Clinical Hematology (3rd edition), A. Victor Hoffbrand and John E. Pettit (eds.) (Harcourt Publishers Ltd., 2000). See, in particular, the lists in FIG. 11.57, 11.58 and 11.59. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphacytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

Indolent lymphoma is a slow-growing, incurable disease in which the average patient survives between six and 10 years following numerous periods of remission and relapse. In one embodiment, the humanized CD20 binding antibodies or functional fragments thereof are used to treat indolent NHL.

The present humanized 2H7 antibodies or functional fragments thereof are useful as a single-agent treatment in, e.g., for relapsed or refractory low-grade or follicular, CD20-positive, B-cell NHL, or can be administered to patients in conjunction with other drugs in a multi drug regimen.

In specific embodiments, the humanized CD20 binding antibodies and functional fragments thereof are used to treat non-Hodgkin's lymphoma (NHL), lymphocyte predominant Hodgkin's disease (LPHD), small lymphocytic lymphoma (SLL), chronic lymphocytic leukemia (CLL).

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions and responses, allergic reaction, eczema including allergic or atopic eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa/periarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barré syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, lymphadenitis, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In specific embodiments, the humanized 2H7 antibodies and functional fragments thereof are used to treat rheumatoid arthritis and juvenile rheumatoid arthritis, systemic lupus erythematosus (SLE) including lupus nephritis, Wegener's disease, inflammatory bowel disease, ulcerative colitis, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, ANCA associated vasculitis, diabetes mellitus, Reynaud's syndrome, Sjorgen's syndrome, Neuromyelitis Optica (NMO) and glomerulonephritis.

“Treating” or “treatment” or “alleviation” refers to therapeutic treatment wherein the object is to slow down (lessen) if not cure the targeted pathologic condition or disorder or prevent recurrence of the condition. A subject is successfully “treated” for an autoimmune disease or a CD20 positive B cell malignancy if, after receiving a therapeutic amount of a humanized CD20 binding antibody of the invention according to the methods of the present invention, the subject shows observable and/or measurable reduction in or absence of one or more signs and symptoms of the particular disease. For example, for cancer, significant reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In preferred embodiments, treatment with the antibodies of the invention is effective to result in the cancer patients being progression-free in the cancer 4 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

A “therapeutically effective amount” refers to an amount of an antibody or a drug effective to “treat” a disease or disorder in a subject. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See preceding definition of “treating”. In the case of an autoimmune disease, the therapeutically effective amount of the antibody or other drug is effective to reduce the signs and symptoms of the disease.

The parameters for assessing efficacy or success of treatment of the neoplasm will be known to the physician of skill in the appropriate disease. Generally, the physician of skill will look for reduction in the signs and symptoms of the specific disease. Parameters can include median time to disease progression, time in remission, stable disease.

The following references describe lymphomas and CLL, their diagnoses, treatment and standard medical procedures for measuring treatment efficacy. Canellos G P, Lister, T A, Sklar J L: The Lymphomas. W.B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D: Chronic Lymphocytic Leukemia, Chap. 72, pp 1350-1362, in: Hematology, Basic Principles and Practice, 3rd ed. Hoffman et al. (editors). Churchill Livingstone, Philadelphia, 2000.

The parameters for assessing efficacy or success of treatment of an autoimmune or autoimmune related disease will be known to the physician of skill in the appropriate disease. Generally, the physician of skill will look for reduction in the signs and symptoms of the specific disease. The following are by way of examples.

In one embodiment, the humanized 2H7 antibodies and specifically hu2H7.v511 and functional fragments thereof are used to treat rheumatoid arthritis.

RA is a debilitating autoimmune disease that affects more than two million Americans and hinders the daily activities of sufferers. RA occurs when the body's own immune system inappropriately attacks joint tissue and causes chronic inflammation that destroys healthy tissue and damage within the joints. Symptoms include inflammation of the joints, swelling, stiffness, and pain. Additionally, since RA is a systemic disease, it can have effects in other tissues such as the lungs, eyes and bone marrow. There is no known cure. Treatments include a variety of steroidal and non-steroidal anti-inflammatory drugs, immunosuppressive agents, disease-modifying anti-rheumatic drugs (DMARDs), and biologics. However, many patients continue to have an inadequate response to treatment.

The antibodies can be used as first-line therapy in patients with early RA (i.e., methotrexate (MTX) naive) and as monotherapy, or in combination with, e.g., MTX or cyclophosphamide. Or, the antibodies can be used in treatment as second-line therapy for patients who were DMARD and/or MTX refractory, and as monotherapy or in combination with, e.g., MTX. The humanized CD20 binding antibodies are useful to prevent and control joint damage, delay structural damage, decrease pain associated with inflammation in RA, and generally reduce the signs and symptoms in moderate to severe RA. The RA patient can be treated with the humanized CD20 antibody prior to, after or together with treatment with other drugs used in treating RA (see combination therapy below). In one embodiment, patients who had previously failed disease-modifying antirheumatic drugs and/or had an inadequate response to methotrexate alone are treated with a humanized CD20 binding antibody of the invention. In one embodiment of this treatment, the patients are in a 17-day treatment regimen receiving humanized CD20 binding antibody alone (Ig i.v. infusions on days 1 and 15); CD20 binding antibody plus cyclophosphamide (750 mg i.v. infusion days 3 and 17); or CD20 binding antibody plus methotrexate.

One method of evaluating treatment efficacy in RA is based on American College of Rheumatology (ACR) criteria, which measures the percentage of improvement in tender and swollen joints, among other things. The RA patient can be scored at for example, ACR 20 (20 percent improvement) compared with no antibody treatment (e.g, baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire, and
5. acute phase reactants, CRP or ESR.
   The ACR 50 and 70 are defined analogously. Preferably, the patient is administered an amount of a CD20 binding antibody of the invention effective to achieve at least a score of ACR 20, preferably at least ACR 30, more preferably at least ACR50, even more preferably at least ACR70, most preferably at least ACR 75 and higher.

Psoriatic arthritis has unique and distinct radiographic features. For psoriatic arthritis, joint erosion and joint space narrowing can be evaluated by the Sharp score as well. The humanized CD20 binding antibodies of the invention can be used to prevent the joint damage as well as reduce disease signs and symptoms of the disorder.

Yet another aspect of the invention is a method of treating SLE or lupus nephritis by administering to a subject suffering from the disorder, a therapeutically effective amount of a humanized CD20 binding antibody of the invention. SLEDAI scores provide a numerical quantitation of disease activity. The SLEDAI is a weighted index of 24 clinical and laboratory parameters known to correlate with disease activity, with a numerical range of 0-103. see Bryan Gescuk & John Davis, “Novel therapeutic agent for systemic lupus erythematosus” in Current Opinion in Rheumatology 2002, 14:515-521. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC).

Spondyloarthropathies are a group of disorders of the joints, including ankylosing spondylitis, psoriatic arthritis and Crohn's disease. Treatment success can be determined by validated patient and physician global assessment measuring tools.

Treatment efficacy for psoriasis is assessed by monitoring changes in clinical signs and symptoms of the disease including Physician's Global Assessment (PGA) changes and Psoriasis Area and Severity Index (PASI) scores, Psoriasis Symptom Assessment (PSA), compared with the baseline condition. The psoriasis patient treated with a humanized CD20 binding antibody of the invention such as hu2H7.v511 can be measured periodically throughout treatment on the Visual analog scale used to indicate the degree of itching experienced at specific time points.

Patients may experience an infusion reaction or infusion-related symptoms with their first infusion of a therapeutic antibody. These symptoms vary in severity and generally are reversible with medical intervention. These symptoms include but are not limited to, flu-like fever, chills/rigors, nausea, urticaria, headache, bronchospasm, angioedema. It would be desirable for the disease treatment methods of the present invention to minimize infusion reactions. To alleviate or minimize such adverse events, the patient may receive an initial conditioning or tolerizing dose(s) of the antibody followed by a therapeutically effective dose. The conditioning dose(s) will be lower than the therapeutically effective dose to condition the patient to tolerate higher dosages.

Dosing

Depending on the indication to be treated and factors relevant to the dosing that a physician of skill in the field would be familiar with, the antibodies of the invention will be administered at a dosage that is efficacious for the treatment of that indication while minimizing toxicity and side effects. The desired dosage may depend on the disease and disease severity, stage of the disease, level of B cell modulation desired, and other factors familiar to the physician of skill in the art.

For treatment of an autoimmune disease, it may be desirable to modulate the extent of B cell depletion depending on the disease and/or the severity of the condition in the individual patient, by adjusting the dosage of humanized 2H7 antibody. B cell depletion can but does not have to be complete. Or, total B cell depletion may be desired in initial treatment but in subsequent treatments, the dosage may be adjusted to achieve only partial depletion. In one embodiment, the B cell depletion is at least 20%, i.e., 80% or less of CD20 positive B cells remain as compared to the baseline level before treatment. In other embodiments, B cell depletion is 25%, 30%, 40%, 50%, 60%, 70% or greater. Preferably, the B cell depletion is sufficient to halt progression of the disease, more preferably to alleviate the signs and symptoms of the particular disease under treatment, even more preferably to cure the disease.

The Genentech and Biogen Idec clinical investigations have evaluated the therapeutic effectiveness of treatment of autoimmune diseases using doses of anti-CD20 (hu2H7.v16 and Rituximab) ranging from as low as 10 mg up to a dose of 1 g (see under Background section for Rituximab studies; and WO 04/056312, Example 16). In general, the antibodies were administered in these clinical investigations in two doses, spaced about two weeks apart. Examples of regimens studied in the clinical investigations include, for humanized CD20 antibody 2H7.v16 in rheumatoid arthritis at 2×10 mg (means 2 doses at ˜10 mg per dose; total dose of ˜10.1 mg/m² for a 70 kg, 67 inch tall patient), 2×50 mg (total dose of 55 mg/m² for a 70 kg, 67 in tall patient), 2×200 mg (total dose of 220 mg/m² for a 70 kg, 67 in tall patient), 2×500 mg (total dose of ˜550 mg/m2 for a 70 kg, 67 in tall patient) and 2×1000 mg (total dose of ˜1100 mg/m2 for a 70 kg, 67 in tall patient); and for Rituxan, 2×500 mg (total dose of ˜550 mg/m2 for a 70 kg, 67 in tall patient), 2×1000 mg (total dose of ˜1100 mg/m2 for a 70 kg, 67 in tall patient). At each of these doses, substantial depletion of circulating B-lymphocytes was observed following the administration of the first dose of the antibody.

In the present methods of treating autoimmune diseases and of depleting B cells in a patient having an autoimmune disease, in one embodiment, the patient is administered humanized 2H7.v511 antibody at a flat dose in the range of 0.1 mg to 1000 mg. We have found that at flat doses of less than 300 mg, even at 10 mg, substantial B cell depletion is achieved. Thus, in the present B cell depletion and treatment methods in different embodiments, hu2H7.v511 antibody is administered at dosages of 0.1, 0.5, 1, 5, 10, 15, 20 25, 30, 40, 50, 75, 100, 125, 150, 200, or 250 mg. Lower doses e.g., at 20 mg, 10 mg or lower can be used if partial or short term B cell depletion is the objective.

For the treatment of a CD20 positive cancer, it may be desirable to maximize the depletion of the B cells which are the target of the anti-CD20 antibodies of the invention. Thus, for the treatment of a CD20 positive B cell neoplasm, it is desirable that the B cell depletion be sufficient to at least prevent progression of the disease which can be assessed by the physician of skill in the art, e.g., by monitoring tumor growth (size), proliferation of the cancerous cell type, metastasis, other signs and symptoms of the particular cancer. Preferably, the B cell depletion is sufficient to prevent progression of disease for at least 2 months, more preferably 3 months, even more preferably 4 months, more preferably 5 months, even more preferably 6 or more months. In even more preferred embodiments, the B cell depletion is sufficient to increase the time in remission by at least 6 months, more preferably 9 months, more preferably one year, more preferably 2 years, more preferably 3 years, even more preferably 5 or more years. In a most preferred embodiment, the B cell depletion is sufficient to cure the disease. In preferred embodiments, the B cell depletion in a cancer patient is at least about 75% and more preferably, 80%, 85%, 90%, 95%, 99% and even 100% of the baseline level before treatment.

Examples of dosing regimens and dosages of hu2H7 antibodies including v16 and v511 for clinical trials in the treatment of NHL are described under Experimental Examples 18-20 below.

Doses at mg/dose of 50, 75, 100, 125, 150, 200, 250, 300, 350 mg/dose can also be used in maintenance therapy for B cell malignancies such as NHL.

The frequency of dosing can vary depending on several factors. The patient will be administered at least 2 doses of the humanized 2H7 CD20 binding antibody, and in different embodiments may receive 24, 2-8 doses, 2-10 doses. Typically, the 2 doses are administered within a month, generally 1, 2 or 3 weeks apart. Depending on the level of improvement in the disease or recurrence, further doses can be administered over the course of the disease or as disease maintenance therapy.

Patients having an autoimmune disease or a B cell malignancy for whom one or more current therapies were ineffective, poorly tolerated, or contraindicated can be treated using any of the dosing regimens of the present invention. For example, the invention contemplates the present treatment methods for RA patients who have had an inadequate response to tumor necrosis factor (TNF) inhibitor therapies or to disease-modifying anti-rheumatic drugs (DMARD) therapy.

In another embodiment, treatment at the low dosages 200 mg/dose or less is useful in maintenance therapy.

In one embodiment, the present dosages and dosing regimen are used in treating rheumatoid arthritis (RA).

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Routes of Administration

The humanized 2H7 antibodies are administered to a human patient in accord with known methods, such as by intravenous administration, e.g., as a bolus or by continuous infusion over a period of time, by subcutaneous, intramuscular, intraperitoneal, intracerobrospinal, intra-articular, intrasynovial, intrathecal, or inhalation routes, generally by intravenous or subcutaneous administration.

In on embodiment, the humanized 2H7 antibody is administered by intravenous infusion with 0.9% sodium chloride solution as an infusion vehicle.

In another embodiment, the humanized 2H7 antibody is administered by subcutaneous injection.

Combination Therapy

In treating the B cell neoplasms described above, the patient can be treated with the humanized 2H7 antibodies of the present invention in conjunction with one or more therapeutic agents such as a chemotherapeutic agent in a multidrug regimen. The humanized 2H7 antibody can be administered concurrently, sequentially, or alternating with the chemotherapeutic agent, or after non-responsiveness with other therapy. Standard chemotherapy for lymphoma treatment may include cyclophosphamide, cytarabine, melphalan and mitoxantrone plus melphalan. CHOP is one of the most common chemotherapy regimens for treating Non-Hodgkin's lymphoma. The following are the drugs used in the CHOP regimen: cyclophosphamide (brand names cytoxan, neosar); adriamycin (doxorubicin/hydroxydoxorubicin); vincristine (Oncovin); and prednisolone (sometimes called Deltasone or Orasone). In particular embodiments, the CD20 binding antibody is administered to a patient in need thereof in combination with one or more of the following chemotherapeutic agents of doxorubicin, cyclophosphamide, vincristine and prednisolone. In a specific embodiment, a patient suffering from a lymphoma (such as a non-Hodgkin's lymphoma) is treated with a humanized 2H7 antibody of the present invention in conjunction with CHOP (cyclophosphamide, doxorubicin, vincristine and prednisone) therapy. In another embodiment, the cancer patient can be treated with a humanized 2H7 CD20 binding antibody of the invention in combination with CVP (cyclophosphamide, vincristine, and prednisone) chemotherapy. In a specific embodiment, the patient suffering from CD20-positive NHL is treated with humanized 2H7.v511 in conjunction with CVP. In a specific embodiment of the treatment of CLL, the hu2H7.v511 antibody is administered in conjunction with chemotherapy with one or both of fludarabine and cytoxan.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK-286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gammall and calicheamicin omegail (see, e.g., Agnew, Chem. Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX™, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxoids and taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON®) toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In treating the autoimmune diseases or autoimmune related conditions described above, the patient can be treated with one or more hu2H7 antibodies such as hu2H7.v511, in conjunction with a second therapeutic agent, such as an immunosuppressive agent, such as in a multi drug regimen. The hu2H7 antibody can be administered concurrently, sequentially or alternating with the immunosuppressive agent or upon non-responsiveness with other therapy. The immunosuppressive agent can be administered at the same or lesser dosages than as set forth in the art. The preferred adjunct immunosuppressive agent will depend on many factors, including the type of disorder being treated as well as the patient's history.

“Immunosuppressive agent” as used herein for adjunct therapy refers to substances that act to suppress or mask the immune system of a patient. Such agents would include substances that suppress cytokine production, down regulate or suppress self-antigen expression, or mask the MHC antigens. Examples of such agents include steroids such as glucocorticosteroids, e.g., prednisone, methylprednisolone, and dexamethasone; 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; cytokine or cytokine receptor antagonists including anti-interferon-γ, -β, or -α antibodies; anti-tumor necrosis factor-α antibodies; anti-tumor necrosis factor-β, antibodies; anti-interleukin-2 antibodies and anti-IL-2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-β; streptodornase; RNA or DNA from the host; FK506; RS-61443; deoxyspergualin; rapamycin; T-cell receptor (U.S. Pat. No. 5,114,721); T-cell receptor fragments (Offner et al., Science 251:430-432 (1991); WO 90/11294; and WO 91/01133); and T cell receptor antibodies (EP 340,109) such as T10B9.

For the treatment of rheumatoid arthritis, the patient can be treated with a hu2H7 antibody in conjunction with any one or more of the following drugs: DMARDS (disease-modifying anti-rheumatic drugs (e.g., methotrexate), NSAI or NSAID (non-steroidal anti-inflammatory drugs), HUMIRA™ (adalimumab; Abbott Laboratories), ARAVA® (leflunomide), REMICADE® (infliximab; Centocor Inc., of Malvern, Pa.), ENBREL (etanercept; Immunex, WA), COX-2 inhibitors. DMARDs commonly used in RA are hydroxycloroquine, sulfasalazine, methotrexate, leflunomide, etanercept, infliximab, azathioprine, D-penicillamine, Gold (oral), Gold (intramuscular), minocycline, cyclosporine, Staphylococcal protein A immunoadsorption. Adalimumab is a human monoclonal antibody that binds to TNFα. Infliximab is a chimeric monoclonal antibody that binds to TNFα. Etanercept is an “immunoadhesin” fusion protein consisting of the extracellular ligand binding portion of the human 75 kD (p75) tumor necrosis factor receptor (TNFR) linked to the Fc portion of a human IgG1. For conventional treatment of RA, see, e.g., “Guidelines for the management of rheumatoid arthritis” Arthritis & Rheumatism 46(2): 328-346 (February, 2002). In a specific embodiment, the RA patient is treated with a hu2H7 CD20 antibody of the invention in conjunction with methotrexate (MTX). An exemplary dosage of MTX is about 7.5-25 mg/kg/wk. MTX can be administered orally and subcutaneously.

For the treatment of ankylosing spondylitis, psoriatic arthritis and Crohn's disease, the patient can be treated with a CD20 binding antibody of the invention in conjunction with, for example, Remicade® (infliximab; from Centocor Inc., of Malvern, Pa.), ENBREL (etanercept; Immunex, WA).

Treatments for SLE include high-dose corticosteroids and/or cyclophosphamide (HDCC).

For the treatment of psoriasis, patients can be administered a CD20 binding antibody in conjunction with topical treatments, such as topical steroids, anthralin, calcipotriene, clobetasol, and tazarotene, or with methotrexate, retinoids, cyclosporine, PUVA and UVB therapies. In one embodiment, the psoriasis patient is treated with a CD20 binding antibody sequentially or concurrently with cyclosporine.

To minimize toxicity, the traditional systemic therapies can be administered in rotational, sequential, combinatorial, or intermittent treatment regimens, or lower dosage combination regimens with the hu2H7 CD20 binding antibody compositions at the present dosages.

### Pharmaceutical Formulations

Therapeutic formulations of the hu2H7 CD20-binding antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary hu2H7 antibody formulations are described in WO98/56418, expressly incorporated herein by reference. Another formulation is a liquid multidose formulation comprising the hu2H7 antibody at 40 mg/mL, 25 mM acetate, 150 mM trehalose, 0.9% benzyl alcohol, 0.02% polysorbate 20 at pH 5.0 that has a minimum shelf life of two years storage at 2-8° C. Another anti-CD20 antibody formulation of interest comprises 10 mg/mL antibody in 9.0 mg/mL sodium chloride, 7.35 mg/mL sodium citrate dihydrate, 0.7 mg/mL polysorbate 80, and Sterile Water for Injection, pH 6.5. Yet another aqueous pharmaceutical formulation comprises 10-30 mM sodium acetate from about pH 4.8 to about pH 5.5, preferably at pH5.5, polysorbate as a surfactant in a an amount of about 0.01-0.1% v/v, trehalose at an amount of about 2-10% w/v, and benzyl alcohol as a preservative (U.S. Pat. No. 6,171,586). Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

One formulation for the humanized 2H7.v511 variant is antibody at 12-14 mg/mL in 10 mM histidine, 6% sucrose, 0.02% polysorbate 20, pH 5.8. In a specific embodiment, 2H7 variants and in particular 2H7.v511 is formulated at 20 mg/mL antibody in 10 mM histidine sulfate, 60 mg/ml sucrose, 0.2 mg/ml polysorbate 20, and Sterile Water for Injection, at pH5.8.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### Articles of Manufacture and Kits

Another embodiment of the invention is an article of manufacture containing materials useful for the treatment of autoimmune diseases and related conditions and CD20 positive cancers such as non-Hodgkin's lymphoma. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a hu2H7 antibody, e.g., hu2H7.v511 of the invention. The label or package insert indicates that the composition is used for treating the particular condition. The label or package insert will further comprise instructions for administering the antibody composition to the patient.

Package insert refers to instructions customarily included in commercial packages of therapeutic products, that contain information about the indications, usage, dosage, administration, contraindications and/or warnings concerning the use of such therapeutic products. In one embodiment, the package insert indicates that the composition is used for treating non-Hodgkins' lymphoma.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for B-cell killing assays, as a positive control for apoptosis assays, for purification or immunoprecipitation of CD20 from cells. For isolation and purification of CD20, the kit can contain a hu2H7.v511 antibody coupled to beads (e.g., sepharose beads). Kits can be provided which contain the antibodies for detection and quantitation of CD20 in vitro, e.g. in an ELISA or a Western blot. As with the article of manufacture, the kit comprises a container and a label or package insert on or associated with the container. The container holds a composition comprising at least one anti-CD20 antibody of the invention. Additional containers may be included that contain, e.g., diluents and buffers, control antibodies. The label or package insert may provide a description of the composition as well as instructions for the intended in vitro or diagnostic use.

### EXPERIMENTAL EXAMPLES

### Example 1

### Conversion of an Existing Cell Line to a Less-Fucosylated Cell Line

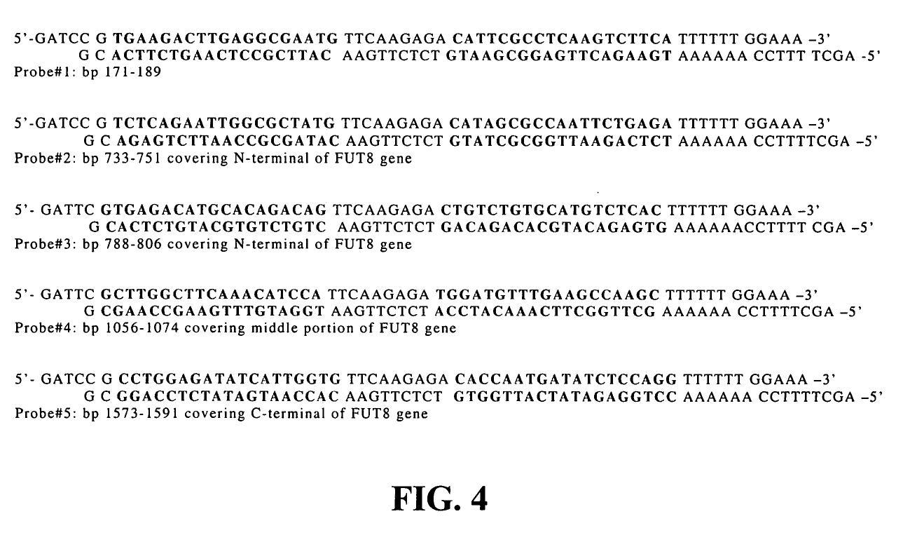

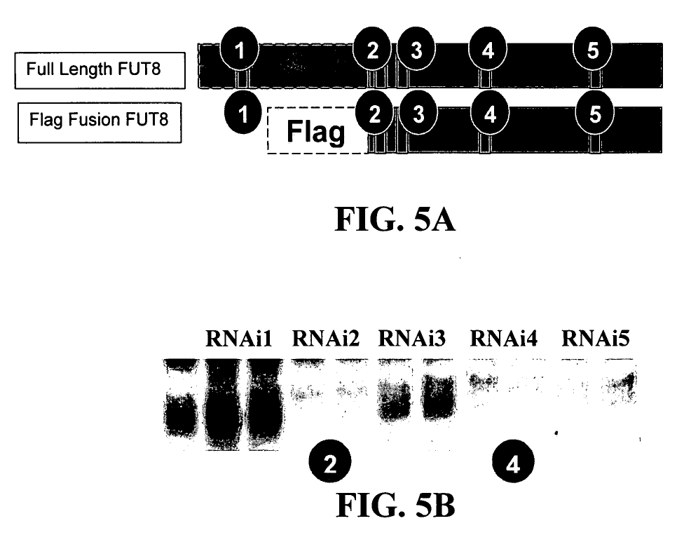

In order to achieve high yields of non-fucosylated antibodies in CHO cells, RNAi approach was employed to knockdown the expression of FUT8 gene. pSilencer 3.1-H1-Puro plasmid from Ambion, Inc. (Austin, Tex.) was used to produce short hairpin siRNA consisting of 19 nt (nucleotide) sense siRNA sequence specific to the gene of FUT8, linked to its reverse complementary antisense siRNA sequence by a short spacer (9 nt hairpin loop), followed by 5-6 U's at 3′ end (FIG. 3). The method used to design siRNA probes to target the CHO FUT8 gene was described by Elbashir et al (2002). Five different siRNA probes were designed (probe #1-5) to target the different regions based on the available CHO FUT8 DNA sequence (FIG. 4). Probe 1 (SEQ ID NO.3 and NO.4); Probe 2 (SEQ ID NO.5 and NO.6); Probe 3 (SEQ ID NO.7 and NO.37); Probe 4 (SEQ ID NO.38 and NO.39); Probe 5 (SEQ ID NO.40 and NO.41). The siRNA encoding sequence consisting of 19 nt sense sequence linked by the spacer to the antisense sequence and 5-6 U's is SEQ ID NO. 42 in probe #2 (positions 7 to 59 in SEQ ID 5) and SEQ ID NO. 43 in probe #4. Probes 1-5 correspond to RNAi 1-5 in FIG. 5B. The five siRNA probes were constructed using annealed synthetic oligonucleotides independently cloned into the pSilencer 3.1-H1-Puro plasmid.

To test the efficacy of these RNAi probes, a FLAG-tagged FUT8 fusion protein was constructed using the CHO FUT8 partial DNA sequence from Genbank (accession no. P_AAC63891). A 3′ 0.98 kb fragment of the FUT8 coding sequence was cloned by reverse transcription polymerase chain reaction (RT-PCR) using total RNA purified from CHO cells and FUT8 primers and the resulting PCR fragment was fused with 5′ FLAG tag sequence. An 8 amino acid Flag tag (metAspTyrLysAspAspAspAspLys—SEQ ID NO. ______) was added to the 5′ end of the isolated partial cDNA sequence. The tagged FUT8 fragment was cloned into an expression vector. The RNAi probe plasmid and flag-tagged FUT8 plasmid were cotransfected into CHO cells. Cell lysate was extracted 24 hours after transfection and the FUT8 fusion protein level was analyzed by anti-flag M2 antibody (Sigma, Mo.) by immunoblotting. In the presence of RNAi probes, the fusion protein expression was significantly inhibited in four out of the five cases (FIG. 5).

The ability of these probes to cleave the FUT8 transcript was tested by transient cotransfection of each siRNA expression plasmid with the Flag-tagged FUT8 plasmid into CHO cells. Cells were lysed 24 hours after transfection and the cell lysate was analyzed by western blot with anti-FlagM2 antibody (Sigma, Mo.).

RNAi (probe 1) transfected cells, as expected, showed strong expression of the Flag-tagged FUT8 product since the Flag-tagged FUT8 fusion protein does not contain the sequence targeted by this probe (FIG. 5A, 5B). In contrast, siRNA probes 2 (RNAi2) through 5 all have various degrees of inhibitory effects on Flag-tagged FUT8 fusion protein expression (FIG. 5B). Probe#2 and #4 showed the best inhibitory effect and were chosen for further evaluation.

### Example 2

### Fucose Content of Stably Expressed Antibodies Manipulated by Transient siRNA Expression

RNAi2 and RNAi4 plasmids were transiently transfected into a previously established stable CHO cell line expressing a humanized anti-CD20 antibody, 2H7.v16 (clone #60). The transfected cells were then separately seeded into 250 ml spinner vessels in serum free medium for antibody production.

The expressed and secreted 2H7.v16 antibody in the harvested cell culture fluid was purified by a protein A column and N-linked oligosaccharides were analyzed for fucose content by matrix-assisted laser desorption/ionization time-of-flight mass spectral analysis (MALDI-TOF) as described in Papac et al., 1998. The antibody was also assayed in a FcγR binding assay (described below). There are 3 groups of human Fcγ receptors: FcγRI, FcγRII, and FcγRIII. Some of these have a functional allelic polymorphism generating allotypes with different receptor properties (Dijstelbloem et al., 1999; Lehrnbecher et al., 1999). FcγRIII (F158) has phenylalanine at position 158 and has a lower binding affinity for the Fc region of human IgGs than FcγRIII (V158) which has a valine at position 158 (Shields et al., 2001 and 2002).

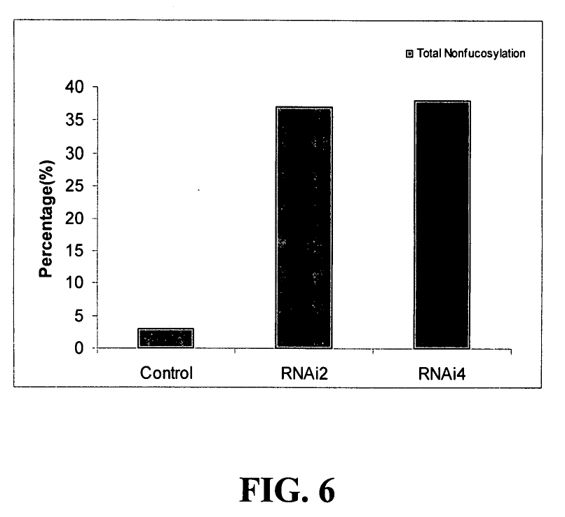

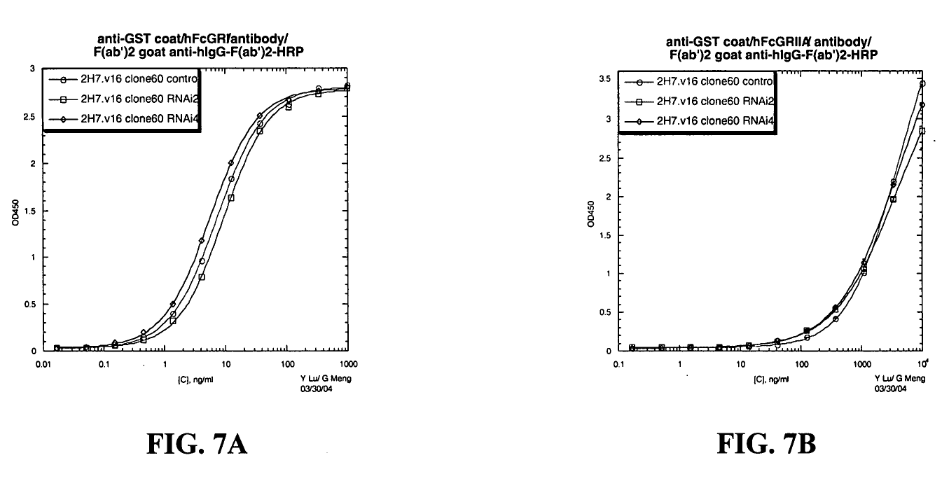

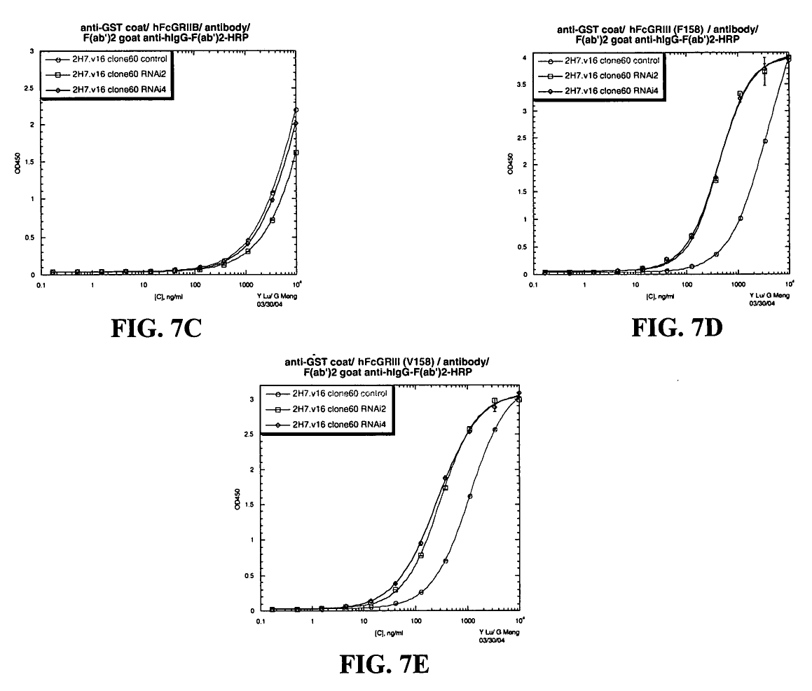

The RNAi transiently transfected cells produced about 35 to 37% nonfucosylated 2H7 antibody as shown in FIG. 6. Compared with the 2H7 control cell line (transfected with irrelevant RNAi plasmid) which had about 2 to 4% nonfucosylated antibody, a level typical of antibodies generated from regular CHO cells, the 2H7 antibody pool with 35% to 37% nonfucosylation showed a 6 and 4 fold increase in binding affinity toward FcγRIII (F158 allele) and FcγRIII (V158 allele), respectively (FIG. 7D, 7E). No effect was seen with other Fc receptors (e.g., FcγRI and FcγRII—see FIG. 7A, 7B, 7C). Glycans isolated from antibodies produced from both RNAi plasmid transfected and mock-transfected cells had similar distributions of galactose contents when structures with no galactose (G0), one galactose (G1) and two galactose (G2) were compared. These data show that the fucose content of antibodies secreted from a stable production cell line can be decreased by transient RNAi plasmid transfection and that the effect does not alter the other main glycan compositions including G0, G1 and G2 distribution.

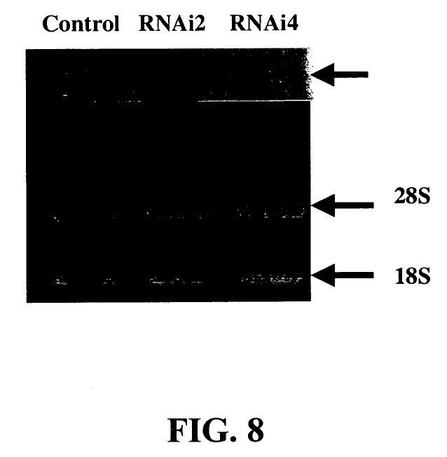

To confirm that the RNAi transfected cells do have less FUT8 RNA expression, a Northern blot was performed using RNA samples extracted from the transfected cells 24 hours after transfection. Total RNA from cells containing a control plasmid (random mouse DNA sequence, no homology to any known mouse proteins) and 2 RNAi plasmids were purified and hybridized with a 300 bp probe. As shown in FIG. 8, the mRNA level was knocked down in two RNAi transfected cells (lanes 2 and 3). This agrees with the immunoblot where lower FUT8 protein amount was detected in two RNAi plasmid transfected cells. The size of CHO FUT8 mRNA is similar to that in rat cells, which is about 3.5 kb. The knock down of endogenous α1,6-fucosyltransferase RNA was further confirmed by quantitative PCR (data not shown).

Since both RNAi2 and RNAi4 constructs can efficiently knock down endogenous FUT8 gene RNA level, only the RNAi4 plasmid was chosen to be used in further stable transfection. The antibody cell line clone 60 which is at 600 nM methotrexate (MTX) resistance and produces over 1.5 g/L in bioreactor was stably transfected with the RNAi4 construct where puromycin gene from the pSilencer plasmid was removed and replaced by hygromycin under the control of SV40 promoter and selected with 500 μg/ml hygromycin. The positive clones were picked into a 96-well tissue culture plate and screened by Taqman for endogenous FUT8 mRNA level. The 4 clones showing different levels of FUT8 mRNA decreasing were scaled up to produce antibody in 250 ml spinners. Antibodies in the HCCF were protein A purified and submitted for fucose content assay and FcγRII binding assays. The results from FIG. 7A-E showed that of the Fcγ receptors tested only FcγRIII binding was affected with lower fucose-containing antibody. Therefore, the antibody products from the stable transfection were submitted for only FcγRIII binding assays.

Analysis of fucose content showed that the 4 lines produced nonfucosylated antibody ranging from 45 to 70% or 80%. Antibodies containing 5 different levels of fucosylation were assayed for their binding to FcγRIII. FcγRIII binding assay showed that there was an increased improvement with low affinity FcγRIII (F158) than FcγRIII (V158) as shown in Table 1. When the fold increase was plotted against the square of the percentage of non-fucosylated material in each antibody sample, a linear relationship was seen for both FcγRIII variants. Intact human IgG1 contains two heavy chains, each with a N-glycosylation site at Asn²⁹⁷ in the CH2 domain of the Fc region. Therefore there are 3 possibilities for the Fc in terms of fucose occupancy of the core carbohydrate structure. One heavy chain is fucosylated and one is not; both heavy chains are fucosylated; or neither heavy chain is fucosylated. The linear relationship between the fold increase in affinity to FcγRIII and the square of the percentage of non-fucosylated glycans indicates that in this case antibody molecules with neither heavy chain fucosylated may provide the major contribution to the improvement of increased FcγRIII binding affinity.

In a further scale up of antibody production by two of the stable transfection clones to a bioreactor, analysis of the fucose content showed that the fucosylation level remained stable over the 79-day period studied, at about 80% nonfucosylation. Antibody titers as well as % G0, G1 and G2 on antibody glycans were also in the expected range at the end of the bioreactor run. Therefore, transfection of RNAi plasmid into an established protein production cell line, antibody producing cell line in this case, is one approach that can be used to generate host cells that produce commercial amounts of a therapeutic antibody with controlled amounts of non-fucosylated carbohydrate.

TABLE 1
FcγRIII bind affinities with antibodies of different fucose contents

| | | FcγRIII(V158) | FcγRIII (F158) |
|---|---|---|---|
| Clones | Non-Fucosylation (%) | Binding (fold) | Binding (fold) |
| Control | 3 | 1 | 1 |
| 5B | 45 | 7.5 | 24.2 |
| 6C | 60 | 10.1 | 34.0 |
| 5F | 70 | 13.7 | 52.4 |
| 7C | 63 | 10.4 | 32.4 |
| Parent | 5 | 1 | 1 |

### Example 3

In this example, we constructed a new version of RNAi plasmid that contains two RNAi transcriptional units, targeting two different regions of FUT8 gene. This plasmid was more potent than the previous version targeting only one region of the gene.

### Example 4

### Generation of New Stable Cell Line with Simultaneous Metabolic Engineering of Fucose Content

### Knockdown of Fucosylation Level

### Materials and Methods

### Cell Culture and Transfection

Chinese Hamster Ovary (CHO) cells were grown in growth medium with 5% FBS (fetal bovine serum) and 1×GHT (glycine, hypoxanthine, and thymidine) at 37° C. For transient transfection, DMRIE-C transfection reagent (Invitrogen) was used. For stable transfection, Lipofectamine 2000 (Invitrogen) was used.

### Selection

After the transfection, cells were centrifuged to collect the pellet. The pellet was then resuspended in medium containing 25 nM methotrexate (MTX). Medium was changed every 3 to 4 days. About 2 weeks after transfection, individual clones were picked and grown in 96-well plates. Usually it takes about 1 week for cells to grow confluent in a 96-well plate.

### Equal Seeding Density Assay

5×10⁴ cells/well were seeded into the 96-well plate. The next day, the growth medium was removed and replaced with the production medium. The day after adding the production medium, the plate was incubated at 33° C. for 5-6 days before the ELISA assay.

### ELISA Assay

When cells are confluent, the growth medium was removed and production medium was added into each well. The day after adding the production medium, the plate was incubated at 33° C. for 5-6 days before the ELISA assay. Typically an ELISA is performed with serial dilutions.

### RNA Analysis

Total RNA was purified with Qiagen's RNA purification kit and quantified by Taqman with gene specific primers and probes.

### Fcγ Receptor Binding Assay—ELISA

MaxiSorp 96-well microwell plates (Nunc, Roskilde, Denmark) were coated with 2 μg/ml anti-GST (clone 8E2.1.1, Genentech), at 100 μl/well in 50 mM carbonate buffer, pH 9.6, at 4° C. overnight. Plates were washed with PBS containing 0.05% polysorbate, pH 7.4 (wash buffer) and blocked with PBS containing 0.5% BSA, pH 7.4, at 150 μl/well. After a one-hour incubation at room temperature, plates were washed with wash buffer. Human FcγRIII was added to the plates at 0.25 μg/ml, 100 μl/well, in PBS containing 0.5% BSA, 0.05% polysorbate 20, pH 7.4. (assay buffer). The plates were incubated for one hour and washed with wash buffer. Antibodies were incubated with goat F(ab′)₂ anti-K (Cappel, ICN Pharmaceuticals, Inc., Aurora, Ohio) at a 1:2 (w/w) ratio for 1 hour to form antibody complexes. Eleven twofold serial dilutions of complexed IgG antibodies (0.85-50000 ng/ml in threefold serial dilution) in assay buffer were added to the plates. After a two-hour incubation, plates were washed with wash buffer. Bound IgG was detected by adding peroxidase labeled goat F(ab′)₂ anti-human IgG F(ab′)₂ (Jackson ImmunoResearch, West Grove, Pa.) at 100 μl/well in assay buffer. After a one-hour incubation, plates were washed with wash buffer and the substrate 3,3′,5,5′-tetramethyl benzidine (TMB) (Kirkegaard & Perry Laboratories) was added at 100 μl/well. The reaction was stopped by adding 1 M phosphoric acid at 100 μl/well. Absorbance was read at 450 nm on a multiskan Ascent reader (Thermo Labsystems, Helsinki, Finland). The absorbance at the midpoint of the standard curve (mid-OD) was calculated. The corresponding concentrations of standard and samples at this mid-OD were determined from the titration curves using a four-parameter nonlinear regression curve-fitting program (KaleidaGraph, Synergy software, Reading, Pa.). The relative activity was calculated by dividing the mid-OD concentration of standard by that of sample.

### Antibody Dependent Cellular Cytotoxicity (ADCC) Assays

One ADCC assay format was as follows. 2H7 IgG variants were assayed for their ability to mediate Natural-Killer cell (NK cell) lysis of WIL2-S cells, a CD20 expressing lymphoblastoid B-cell line, essentially as described (Shields et al., J. Biol. Chem. 276:6591-6604 (2001)) using a lactate dehydrogenase (LDH) readout. NK cells were prepared from 100 mL of heparinized blood, diluted with 100 mL of PBS (phosphate buffered saline), obtained from normal human donors who had been isotyped for FcγRIII, also known as CD16 (Koene et al., Blood 90:1109-1114 (1997)). The NK cells can be from human donors heterozygous for CD16 (F158N158) of homozygous for V158 or F158. The diluted blood was layered over 15 mL of lymphocyte separation medium (ICN Biochemical, Aurora, Ohio) and centrifuged for 20 min at 2000 RPM. White cells at the interface between layers were dispensed to 4 clean 50-mL tubes, which were filled with RPMI medium containing 15% fetal calf serum. Tubes were centrifuged for 5 min at 1400 RPM and the supernatant discarded. Pellets were resuspended in MACS buffer (0.5% BSA, 2 mM EDTA), and NK cells were purified using beads (NK Cell Isolation Kit, 130-046-502) according to the manufacturer's protocol (Miltenyi Biotech,). NK cells were diluted in MACS buffer to 2×10⁶ cells/mL.

Serial dilutions of antibody (0.05 mL) in assay medium (F12/DMEM 50:50 without glycine, 1 mM HEPES buffer pH 7.2, Penicillin/Streptomycin (100 units/mL; Gibco), glutamine, and 1% heat-inactivated fetal bovine serum) were added to a 96-well round-bottom tissue culture plate. WIL2-S cells were diluted in assay buffer to a concentration of 4×10⁵/mL. WIL2-S cells (0.05 mL per well) were mixed with diluted antibody in the 96-well plate and incubated for 30 min at room temperature to allow binding of antibody to CD20 (opsonization).

The ADCC reaction was initiated by adding 0.1 mL of NK cells to each well. In control wells, 2% Triton X-100 was added. The plate was then incubated for 4 h at 37° C. Levels of LDH released were measured using a cytotoxicity (LDH) detection kit (Kit#1644793, Roche Diagnostics, Indianapolis, Ind.) following the manufacturers instructions. 0.1 mL of LDH developer was added to each well, followed by mixing for 10 s. The plate was then covered with aluminum foil and incubated in the dark at room temperature for 15 min. Optical density at 490 nm was then read and use to calculate % lysis by dividing by the total LDH measured in control wells. Lysis was plotted as a function of antibody concentration, and a 4-parameter curve fit (KaleidaGraph) was used to determine EC₅₀ concentrations.

### Matrix-Assisted Laser Desorption/Ionization Time-of-Flight (MALDI-TOF) Mass Spectral Analysis of Asparagine-Linked Oligosaccharides:

N-linked oligosaccharides were released from recombinant glycoproteins using the procedure of Papac et al., Glycobiology 8, 445-454 (1998). Briefly, the wells of a 96 well PVDF-lined microtitre plate (Millipore, Bedford, Mass.) were conditioned with 100 μl methanol that was drawn through the PDVF membranes by applying vacuum to the Millipore Multiscreen vacuum manifold. The conditioned PVDF membranes were washed with 3×250 μl water. Between all wash steps the wells were drained completely by applying gentle vacuum to the manifold. The membranes were washed with reduction and carboxymethylation buffer (RCM) consisting of 6 M guanidine hydrochloride, 360 mM Tris, 2 mM EDTA, pH 8.6. Glycoprotein samples (50 μg) were applied to individual wells, again drawn through the PVDF membranes by gentle vacuum and the wells were washed with 2×50 μl of RCM buffer. The immobilized samples were reduced by adding 50 μl of a 0.1 M dithiothreitol (DTT) solution to each well and incubating the microtitre plate at 37° C. for 1 hr. DTT was removed by vacuum and the wells were washed 4×250 μl water. Cysteine residues were carboxylmethylated by the addition of 50 μl of a 0.1 M iodoacetic acid (IAA) solution which was freshly prepared in 1 M NaOH and diluted to 0.1 M with RCM buffer. Carboxymethylation was accomplished by incubation for 30 min in the dark at ambient temperature. Vacuum was applied to the plate to remove the LAA solution and the wells were washed with 4×250 μl purified water. The PVDF membranes were blocked by the addition of 100 μl of 1% PVP360 (polyvinylpyrrolidine 360,000 MW) (Sigma) solution and incubation for 30 minutes at ambient temperature. The PVP-360 solution was removed by gentle vacuum and the wells were washed 4×250 μl water. The PNGase F (New England Biolabs, Beverly, Mass.) digest solution, 25 μl of a 25 Unit/ml solution in 10 mM Tris acetate, pH 8.3, was added to each well and the digest proceeded for 3 hr at 37° C. After digestion, the samples were transferred to 500 μl Eppendorf tubes and 2.5 μl of a 1.5 M acetic acid solution was added to each sample. The acidified samples were incubated for 2 hr at ambient temperature to convert the oligosaccharides from the glycosylamine to the hydroxyl form. Prior to MALDI-TOF mass spectral analysis, the released oligosaccharides were desalted using a 0.7-ml bed of cation exchange resin (AG50W-X8 resin in the hydrogen form) (Bio-Rad, Hercules, Calif.) slurried packed into compact reaction tubes (US Biochemical, Cleveland, Ohio).

For MALDI-TOF mass spectral analysis of the samples in the positive mode, the desalted oligosaccharides (0.5 μl aliquots) were applied to the stainless target with 0.5 μl of the 2,5 dihydroxybenzoic acid matrix (sDHB) that was prepared by dissolving 2 mg 2,5 dihydroxybenzoic acid with 0.1 mg of 5-methoxysalicylic acid in 1 ml of 1 mM NaCl in 25% aqueous ethanol. The sample/matrix mixture was dried by vacuum. The sample/matrix mixture was vacuum dried and then allowed to absorb atmospheric moisture prior to analysis. Released oligosaccharides were analyzed by MALDI-TOF on a PerSeptive BioSystems Voyager-ELITE mass spectrometer. The mass spectrometer was operated in the positive mode at 20 kV with the linear configuration and utilizing delayed extraction. Data were acquired using a laser power of approximately 1100 and in the data summation mode (240 scans) to improve the signal to noise. The instrument was calibrated with a mixture of standard oligosaccharides and the data was smoothed using a 19 point Savitsky-Golay algorithm before the masses were assigned. Integration of the mass spectral data was achieved using Caesar 7.2 data analysis software package (SciBridge Software).

### Results and Discussion

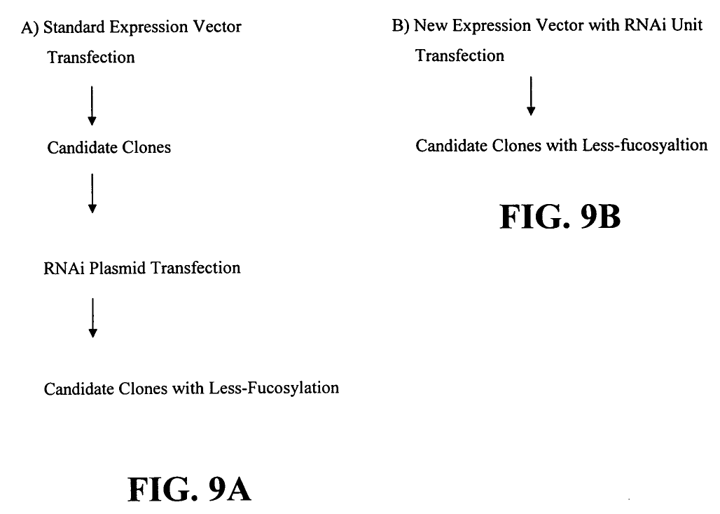

In the previous examples, α-1,6-fucosyltransferase (FUT8) activity was knocked down in 2H7. v16 cell line using RNAi technology. The RNAi targeted an area within the open reading frame (ORF) of FUT8 gene. The less-fucosylated antibodies produced by this cell line displayed higher binding affinity towards FcγRIII receptors, and higher ADCC activity than the highly fucosylated antibodies. FIG. 9A shows the process that was used to develop a less-fucosylated 2H7.v16 cell line. The above process is a two-step approach requiring the existence of a stable antibody producing cell line before RNAi plasmid transfection.

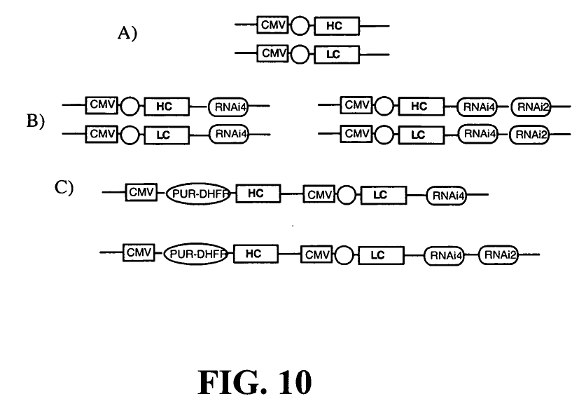

To shorten the time needed for this process, a new, one-step approach was explored where the siRNA unit(s) has been included in the expression plasmid expressing the protein of interest (e.g., antibody), as illustrated in FIG. 9B. First, the expression plasmids containing the antibody expression cassette and RNAi unit(s) were tested to see if antibody and RNAi could be expressed simultaneously in transient transfection. The configuration of the five sets of plasmids transiently transfected is shown in FIG. 10. The proteins expressed from those five sets of plasmids were assayed for fucosylation level.

In Table 2 below, v511 and v114 refer to hu2H7 antibody variants described in Table 3. As shown in Table 2, the antibody from control plasmids containing no RNAi unit has 9% non-fucosylation. The antibodies expressed from the plasmids that contain one RNAi unit have non-fucosylation ranging from 33% to 49%. The antibodies expressed from the plasmids containing two RNAi units have non-fucosylation ranging from 62% to 65%. These results show that addition of two RNAi transcription units on expression plasmids led to the production of antibodies with higher nonfucosylation of 62-65% compared to 33-49% with only one RNAi unit on the expression plasmids, indicating additive effects of the two siRNAi transcripts. The antibody expressed in this example is humanized anti-CD20 antibody 2H7.v511 (also referred to herein as hu2H7.v511) the sequences of which are provided above under CD20 binding antibodies.

TABLE 2

| Plasmid | Nonfucosylation Percentage |
|---|---|
| rkHCv511 + rkLCv114 | 9 |
| rkHCv511RNAi4 + rkLCv114RNAi4 | 49 |
| rkHCv511RNAi2.4 + rkLCv114RNAi2.4 | 62 |
| CMV.PD.v511.RNAi4 | 33 |
| CMV.PD.v511.RNAi2.4 | 65 |

Cells were stably transfected with one of two plasmids, CMV.PD.v511.RNAi4 or CMV.PD.v511.RNAi2.4 (FIG. 10C) and the transfected cells selected with 25 nM methotrexate (MTX). From each transfection, 72 clones were picked and screened for antibody expression. Expression titers are shown in FIG. 11. Clones from the CMV.PD.v511.RNAi2.4 plasmid transfection appeared to have lower titers overall compared to the other two transfections.

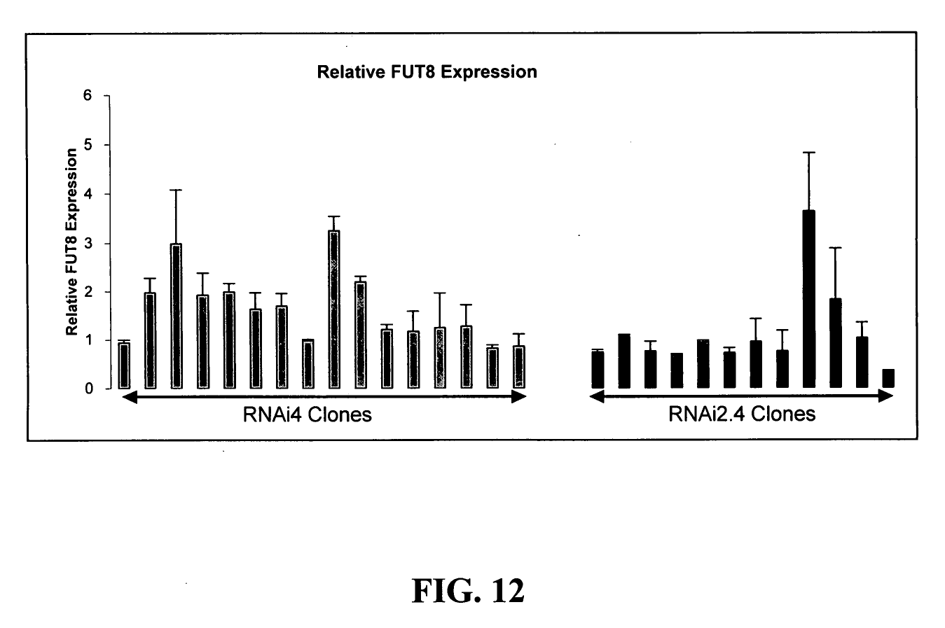

To see if the clones that have good expression titers also have lower fucosylation levels, about 20% of clones with higher expression were analyzed for FUT 8 mRNA expression by Taqman. As shown in FIG. 12, clones from the CMV.PD.v511.RNAi2.4 plasmid transfection generally have lower FUT8 mRNA levels compared with clones from the CMV.PD.v511.RNAi4 plasmid transfection.

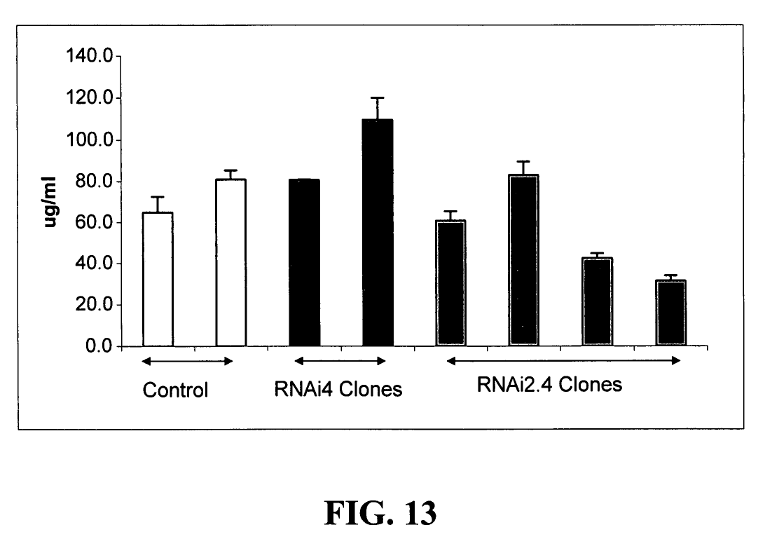

Six clones with lowest FUT8 mRNA expression levels, two from the CMV.PD.v511 RNAi4 plasmid transfection and four from the CMV.PD.v511.RNAi2.4 plasmid transfection, were further evaluated for antibody expression using equal seeding density assay. The results indicated that titers of these six clones are comparable to control 2H7 v511 clones (clone 18 and 63 were from the CMV.PD.v511 plasmid transfection) as shown in FIG. 13. However, CMV.PD.v511.RNAi2.4 clones appear to have lower titer than CMV.PD.v511.RNAi4 clones and control clones.

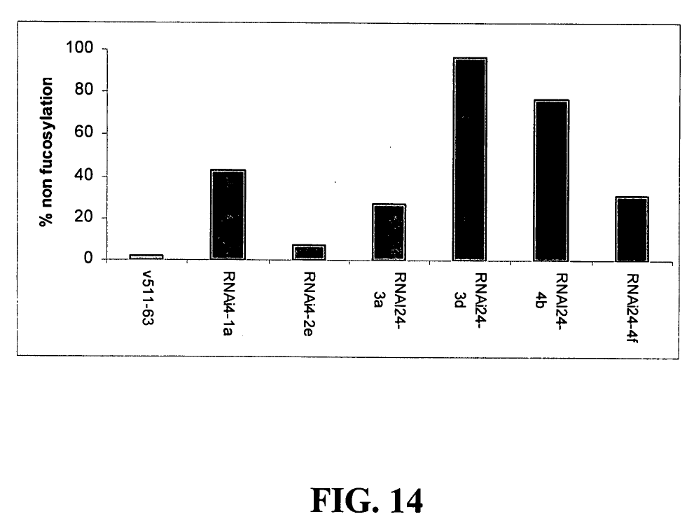

Fucose content of the antibodies produced by the 2H7.v511 clones shown in FIG. 14 was performed by MALDI-TOF mass spectral analysis as described above. It was found that one clone, RNAi24-3d, achieved 94-95% nonfucosylation.

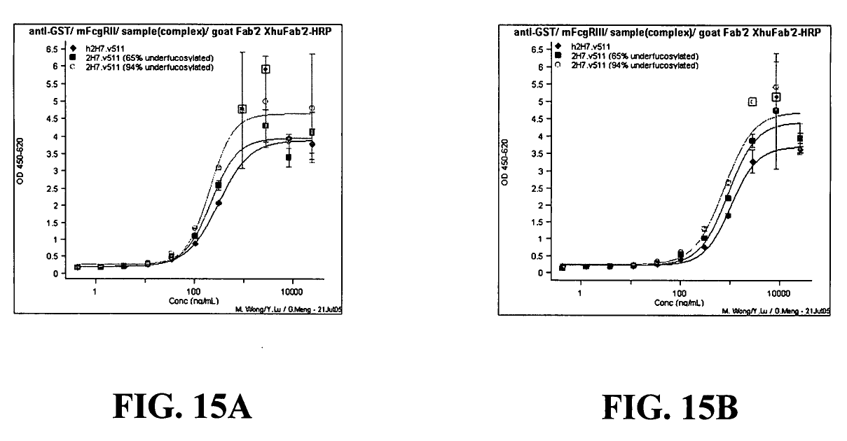

A FcγRIII binding assay was done with antibody 2H7.v511 containing either 65% nonfucosylation (from transient run) or 94-95% nonfucosylation (from best stable clone RNAi2.4-3d). The results are shown in FIG. 15A and FIG. 15B. Compared to control antibody pools, which had about 5% nonfucosylation, the 65% nonfucosylated material showed a moderate 4.8 and 6.2 fold increase in affinity toward the high affinity (V158 allele-FIG. 15B) and low affinity (F158 allele, FIG. 15A) receptors respectively, while the 95% nonfucosylated material showed a 6.8 and 9.8 fold increase in affinity toward the two receptor isotypes.

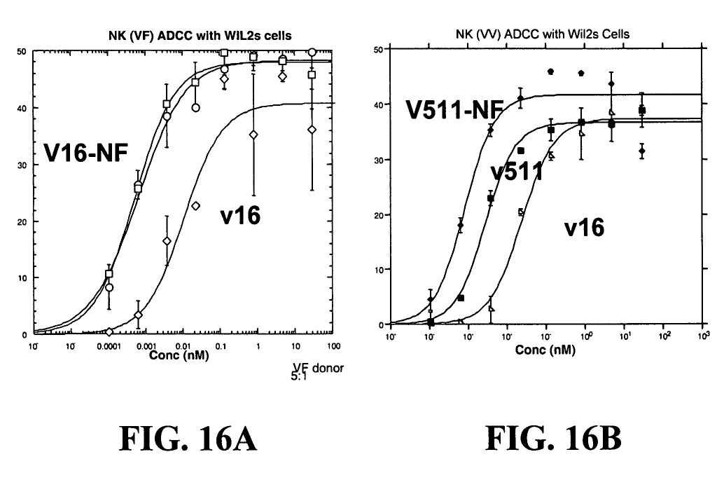

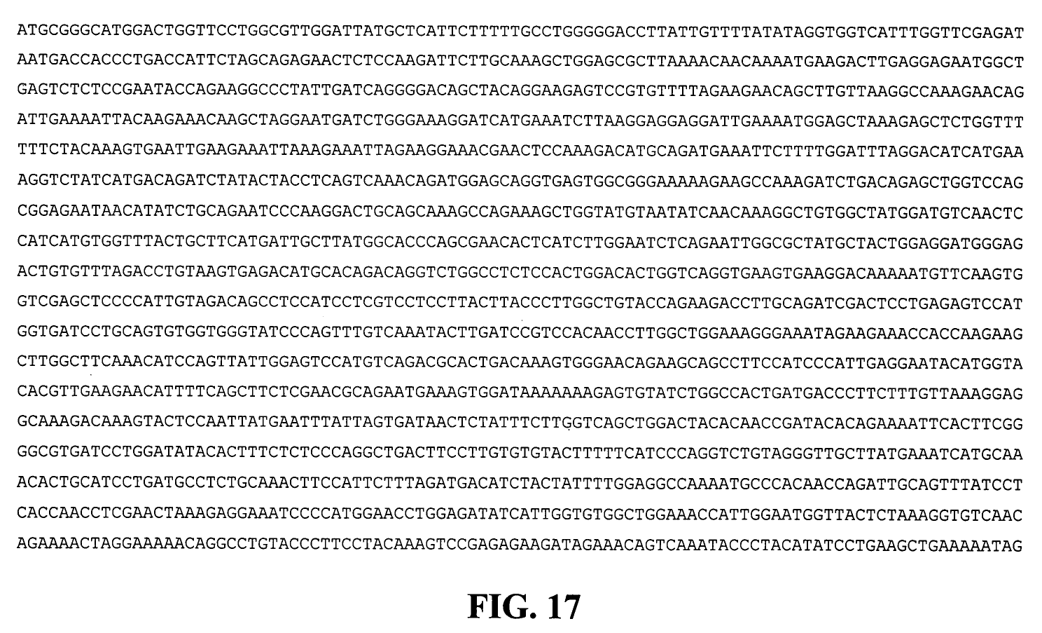

Since nonfucosylated antibodies seem to bind to FcγRIII better, they were tested for their ADCC activities. Materials collected from 2H7.v16 clone 7F (ranging from 60-70% nonfucosylation) and from 2H7.v511 transient transfection (65% nonfucosylation) were used for ADCC activity assay. As seen in FIGS. 16A and 16B, both versions of less fucosylated 2H7 displayed higher ADCC activity compared to their corresponding highly fucosylated counterparts.

Here we described a novel streamlined way to metabolically engineer CHO cells to produce even more highly (as high as 95%) non-fucosylated antibodies by incorporating the antibody heavy chain and light chain transcription units along with 1-2 siRNA transcription unit(s) onto the same plasmid. The two siRNA transcripts used in this approach target different coding regions in the FUT8 gene and are directed by separate Pol III type promoters, H1 and U6.

In summary, we have demonstrated that it is feasible to incorporate RNAi technology into the development of the humanized 2H7 cell lines to knock down fucosylation level. An existing antibody producing cell line was successfully converted to a less-fucosylated cell line. Additionally, simultaneous fucosylation knockdown while generating a new antibody producing cell line was also successfully achieved.

### REFERENCES

References cited within this application, including patents, published applications and other publications, are hereby incorporated by reference.
Anderson, D. C and Krummen, L. (2002) Curr. Opin. Biotech; 13: 117-123.
Boyd, P. N., Lines, A. C. and Patel, A. K. (1995) Mol. Immunol. 32:1311-1318.
Brummelkamp, T. R., Bernards, R. and Agami, R. (2002) Science 296: 550-553.
Cartron, G., Dacheux, L., Salles, G., Solal-Celigny, P., Bardos, P., Colombat, P. and Watier, H. (2002) Blood 99: 754-758.
Chu, L. and Robinson, D. K. (2001) Curr. Opin. Biotechnol. 12:180-187.
Davis, J., Jiang, L., LaBarre, M. J., Andersn, D. and Reff, M. (2001) Biotechnol. Bioeng. 74: 288-294.
Dijstelbloem, H. M., Scheepers, R. H., Oosh, W. W., et al. (1999) Arthritis Rheum. 42:1823-1827.
Dykxhoorn, D. M., Novina, C. D. and Sharp, P. A. (2003) Mol. Cell Biol. 4: 457-467.
Elbashir, S. M., Harborth, J., Weber, K. and Tuschl, T. (2002) Methods 26: 199-213.
Idusogie, E. E., Presta, L. G., Gazzano-Santoro, H., Totpal, K., Wong, P. Y., Ultsch, M., Meng, Y. G. and Mulkerrin, M. G. (2000) J. Immunol. 164: 4178-4184.
Irie, N., Sakai, N., Ueyama, T., Kajimoto, T., Shirai, Y. and Saito, N. (2002) Biochem. Biophys. Res. Commun. 298: 738-743.
Jefferis, R. (2005) Biotechnol. Prog. 21: 11-16.
Katome, T., Obata, T., Matsushima, R., Masuyama, N., Cantley, L. C., Gotoh, Y., Kishi, K., Shiota, H. and Ebina, Y. (2003) J. Bio. Chem. 278: 28312-28323.
Khanzada, U. K., Pardo, O. E., Meier, C., Downward, J., Seckl, M. J. and Arcaro, A. (2006) Oncogene 25:877-887.
Kumpel, B. M., Rademacher, T. W., Rook, G. A., Williams, P. J. and Wilson, I. B. (1994) Hum. Antib. Hybrid. 5: 143-151.
Kumpel, B. M., Wang, Y., Griffiths, H. L., Hadley, A. G. and Rook, G. A. (1995) Hum. Antib. Hybrid. 6: 82-88.
Kunkel, G R and Pederson, I. (1988) Genes Dev 2: 196-204.
Lehrnbecher, T., Foster, C. B., Zhu, S., Leitman, S. F., Goldin, L. R., Huppi, K. and Chanock, S. J. (1999) Blood 94: 4220-4232.
Martinez-Duncker, I. M., Michalski, J. C., Bauvy, C., Candelier, J. J., Mennesson, B., Codogno, P., Oriol, R. and Mollicone, R. (2004) Glycobiology 14: 13-25.
Miyagishi, M. and Taira, K. (2002). Nat. Biotechnol. 19:497-499.
Mori, K., Kamochi, R. K., Ohnuki, N. Y., Wakitani, M., Yamano, K., Imai, H., Kanda, Y., Niwa, R., Lida, S., Uchida, K., Shitara, K. and Satoh, M. (2004) Biotechnol. Bioeng. 88: 901-908.
Niwa, R., Hatanaka, S., Shoji-Hosaka, E., Sakurada, M., Kobayashi, Y., Uehara, A., Yokoi, H., Nakamura, K. and Shitara, K. (2004) Clin. Cancer Res. 10: 6248-6255.
Niwa, R., Hosaka, E. S., Sakurada, M., Shinkawa, T., Uchida, K., Nakamura, K., Matsushima, K., Ueda, R., Hanai, N. and Shitara, K. (2004) Cancer Res. 64: 2127-2133, 2004.
Niwa, R., Sakurada, M., Kobayashi, Y., Uehara, A., Matsushima, K., Ueda, R., Nakamura, K. and Shitara, K. (2005) Clin. Cancer Res. 11: 2327-2336.
Ohnuki, N. Y., Kinoshita, S., Urakubo, M. I., Kusunoki, M., Lida, S., Nakano, R., Wakitani, M., Niwa, R., Sakurada, M., Uchida, K., Shitara, K., and Satoh, M. (2004) Biotech. And Bioengi. 87 (5): 614-622.
Okazaki, A., Hosaka, E. S., Nakamura, K., Wakitani, M., Uchida, K., Kakita, S., Tsumoto, K., Kumagai, I. And Shitara, K. (2004) J. Mol. Biol. 336: 1239-1249.
Papac, D. J., Briggs, J. B., Chin, E. T. and Jones, A. J. S. (1998) Glycobiology 8: 445-454.
Ripka, J., Adamany, A. and Stanley, P. (1986) Archives. Biochem. and Biophy. 249: 533-545.
Saba, J. A., Kunkel, J. P., Jan, D. C. H., Ens, W. E., Standing, K. G., Butler, M., Jamieson, J. C. and Perreault, H. (2002) Anal. Biochem. 305: 16-31.
Schuster, M., Umana, P., Ferrara, C., Brunker, P., Gerdes, C., Waxenecker, G., Weiderkum, S., Schwager, C., Loibner, H., Himmler, G. and Mudde, G. C. (2005) Cancer Res. 65: 7934-7941.
Shields, R. L., Lai, J., Keck, R., O'Connell, L. Y., Hong, K., Meng, Y. G., Weikert, S. H. A. and Presta, L. G. (2002) J. Biol. Chem. 277: 26733-26740.
Shields, R. L., Lai, J., Keck, R., O'Connell, L. Y., Hong, K., Meng, Y. G., Weikert, S. H. and Presta, L. G. (2001) J. Biol. Chem. 276: 6591-6604.
Shinkawa, T., Nakamura, K., Yamane, N., Hosaka, E. S., Kanda, Y., Sakurada, M., Uchida, K., Anazawa, H., Satoh, M., Yamasaki, M., Hanai, N. and Shitara, K. (2003) J. Biol. Chem. 278: 3466-3473.
Sui, G., Soohoo, C., Affar, E. B., Gay, F., Shi, Y., Forrester, W. C. and Shi, Y. (2002) Proc. Natl. Acad. Sci. USA 99: 5515-5520.
Taniguchi, N., Gu, J., Takahashi, M. and Miyoshi, E. (2004) Proc. Japan Acad., Ser. B80: 82-91.
Umana, P., Lean-Mairet, J., Moudry, R., Amstutz, H. and Bailey, J. E. (1999) Nat. Biotechnol. 17: 176-180.
Wang, Y., Fei, D., Vanderlaan, M. and Song, A. (2004) Angiogenesis 7: 335-345.
Weng, W. K. and Levy, R. (2003) J. Clin. Oncol. 21: 3940-3947.
Yamaguchi, Y., Ikeda, Y., Takahashi, T., Ihara, H., Tanaka, T., Sasho, C., Uozumi, N., Yanagidani, S., Inoue, S., Fujii. J. and Taniguchi, N. (2000) Glycobiology 10: 637-643.
Yamane-Ohnuki, N., Kinoshita, S., Urakubo, M. I., Kusunoki, M., Lida, S., Nakano, R., Wakitani, M., Niwa, R., Sakurada, M., Uchida, K., Shitara, K. and Satoh, M. (2004) Biotechnol. Bioeng. 87: 614-622.

1. A method of producing an antibody comprising an IgG Fc in a mammalian host cell while reducing the fucose content of the antibody, comprising introducing simultaneously into the host cell, at least one nucleic acid encoding an antibody and a second nucleic acid encoding at least two siRNAs targeting different coding regions of the FUT8 gene sequence of SEQ ID NO. 1, wherein the siRNAs inhibit the expression of FUT8 and reduce the fucosylation level of the antibody.

2. The method of claim 1 wherein the nucleic acid encoding an antibody encodes both a light (L) chain and a heavy (H) chain of the antibody.

3. The method of claim 2 wherein the nucleic acid encoding the H and L chains of the antibody and the nucleic acid encoding the siRNAs are on the same expression vector.

4. The method of claim 1 wherein the nucleic acid encoding the H chain and the nucleic acid encoding the L chain are on separate expression vectors wherein each of the expression vectors encoding the H and L chain also comprises a nucleic acid encoding at least two siRNAs.

5. The method of claim 1 wherein the two siRNAs are expressed under the control of separate promoters.

6. The method of claim 5 wherein one siRNA is expressed under the Pol III promoter, H1 and the second siRNAi is expressed under the Pol III promoter, U6.

7. The method of claim 1 wherein the first and second siRNA target nucleotide positions 733-751 and 1056-1074, respectively, of the FUT8 gene sequence of SEQ ID NO. 1.

8. The method of claim 1 wherein the host cell is a Chinese Hamster Ovary (CHO) cell or derivative thereof.

9. The method of claim 1 wherein the antibody fucosylation level is reduced by at least 90%.

10. The method of claim 1 wherein the antibody fucosylation level is reduced by at least 95%.

11. The method of claim 1 wherein the antibody is a therapeutic antibody.

12. An antibody produced by the method of claim 1.

13. A method of producing an IgG antibody with improved ADCC, comprising introducing simultaneously into the host cell, at least one nucleic acid encoding an antibody and a second nucleic acid encoding at least two siRNAs targeting different coding regions of the FUT8 gene sequence of SEQ ID NO. 1, wherein the antibody and the siRNAs are expressed in the cell to produce an antibody with reduced fucosylation and increased ADCC activity as compared to the antibody produced in the cell in the absence of the siRNAs.

14. The method of claim 13 wherein the antibody comprises at least one amino acid alteration in the Fc region that improves antibody binding to FcγRIII and/or ADCC.

15. The method of claim 14 wherein the antibody comprises the Fc amino acid substitutions of S298A, E333A, K334A.

16. The method of claim 15 further comprising the Fc amino acid substitution K326A.

17. The method of claim 13 wherein the antibody binds CD20.

18. The method of claim 17 wherein the antibody binds primate CD20.

19. The method of claim 17 wherein the CD20 binding antibody is a human antibody.

20. The method of claim 17 wherein the CD20 binding antibody is a chimeric antibody.

21. The method of claim 20 wherein the chimeric antibody is rituximab.

22. The method of claim 17 wherein the CD20 binding antibody is a humanized antibody.

23. The method of claim 22 wherein the humanized CD 20 binding antibody comprises the VL and VH regions selected from the VL of SEQ ID NO.2 and the VH of SEQ ID NO.8; VL of SEQ ID NO.25 and the VH of SEQ ID NO.22; and VL of SEQ ID NO.25 and the VH of SEQ ID NO.33.

24. The method of claim 22 wherein the humanized CD20 binding antibody comprises the L and H chain having the sequence of SEQ ID NO. 13 and 14, respectively.

25. The method of claim 22 wherein the humanized CD20 binding antibody comprises the L and H chain having the sequence of SEQ ID NO. 26 and SEQ ID NO. 27, respectively.

26. The method of claim 22 wherein the humanized CD 20 binding antibody comprises the L and H chain having the sequence of SEQ ID NO. 26 and SEQ ID NO. 34, respectively.

27. The method of claim 13 wherein the antibody binds BR3.

28. An antibody produced by the method of claim 13.

29. A nucleic acid comprising the sequence of SEQ ID NO. 10 and SEQ ID NO. 11.

30. A composition comprising humanized CD20 binding antibodies having an Fc region, and a carrier, wherein at least 95% of the antibodies in the composition lack fucose.

31. A host cell comprising at least one nucleic acid encoding an antibody and a second nucleic acid encoding at least two siRNAs targeting different coding regions of the FUT8 gene sequence of SEQ ID NO. 1, wherein the host cell expresses the antibody and the siRNAs.


SEQUENCE LISTING (41 sequences)

SEQ ID NO: 1 - DNA, 1728 bp, Cricetulus griseus
atgcgggcatggactggttcctggcgttggattatgctcattctttttgcctgggggaccttattgttttatataggtggtcatttggttcgagataatgaccaccctgaccattctagcagagaactctccaagattcttgcaaagctggagcgcttaaaacaacaaaatgaagacttgaggagaatggctgagtctctccgaataccagaaggccctattgatcaggggacagctacaggaagagtccgtgttttagaagaacagcttgttaaggccaaagaacagattgaaaattacaagaaacaagctaggaatgatctgggaaaggatcatgaaatcttaaggaggaggattgaaaatggagctaaagagctctggttttttctacaaagtgaattgaagaaattaaagaaattagaaggaaacgaactccaaagacatgcagatgaaattcttttggatttaggacatcatgaaaggtctatcatgacagatctatactacctcagtcaaacagatggagcaggtgagtggcgggaaaaagaagccaaagatctgacagagctggtccagcggagaataacatatctgcagaatcccaaggactgcagcaaagccagaaagctggtatgtaatatcaacaaaggctgtggctatggatgtcaactccatcatgtggtttactgcttcatgattgcttatggcacccagcgaacactcatcttggaatctcagaattggcgctatgctactggaggatgggagactgtgtttagacctgtaagtgagacatgcacagacaggtctggcctctccactggacactggtcaggtgaagtgaaggacaaaaatgttcaagtggtcgagctccccattgtagacagcctccatcctcgtcctccttacttacccttggctgtaccagaagaccttgcagatcgactcctgagagtccatggtgatcctgcagtgtggtgggtatcccagtttgtcaaatacttgatccgtccacaaccttggctggaaagggaaatagaagaaaccaccaagaagcttggcttcaaacatccagttattggagtccatgtcagacgcactgacaaagtgggaacagaagcagccttccatcccattgaggaatacatggtacacgttgaagaacattttcagcttctcgaacgcagaatgaaagtggataaaaaaagagtgtatctggccactgatgacccttctttgttaaaggaggcaaagacaaagtactccaattatgaatttattagtgataactctatttcttggtcagctggactacacaaccgatacacagaaaattcacttcggggcgtgatcctggatatacactttctctcccaggctgacttccttgtgtgtactttttcatcccaggtctgtagggttgcttatgaaatcatgcaaacactgcatcctgatgcctctgcaaacttccattctttagatgacatctactattttggaggccaaaatgcccacaaccagattgcagtttatcctcaccaacctcgaactaaagaggaaatccccatggaacctggagatatcattggtgtggctggaaaccattggaatggttactctaaaggtgtcaacagaaaactaggaaaaacaggcctgtacccttcctacaaagtccgagagaagatagaaacagtcaaataccctacatatcctgaagctgaaaaatag

SEQ ID NO: 2 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 3 - DNA, 64 bp, Cricetulus griseus
gatccgtgaagacttgaggcgaatgttcaagagacattcgcctcaagtcttcattttttggaaa

SEQ ID NO: 4 - DNA, 64 bp, Cricetulus griseus
agcttttccaaaaaatgaagacttgaggcgaatgtctcttgaacattcgcctcaagtcttcacg

SEQ ID NO: 5 - DNA, 64 bp, Cricetulus griseus
gatccgtctcagaattggcgctatgttcaagagacatagcgccaattctgagattttttggaaa

SEQ ID NO: 6 - DNA, 64 bp, Cricetulus griseus
agcttttccaaaaaatctcagaattggcgctatgtctcttgaacatagcgccaattctgagacg

SEQ ID NO: 7 - DNA, 63 bp, Cricetulus griseus
gattcgtgagacatgcacagacagttcaagagactgtctgtgcatgtctcacttttttggaaa

SEQ ID NO: 8 - protein, 122 aa, Artificial sequence [sequence omitted]

SEQ ID NO: 9 - protein, 9 aa, Artificial sequence
  sequence is synthesized
MDYKDDDDK

SEQ ID NO: 10 - DNA, 53 bp, Cricetulus griseus
tctcagaattggcgctatgttcaagagacatagcgccaattctgagatttttt

SEQ ID NO: 11 - DNA, 53 bp, Cricetulus griseus
gcttggcttcaaacatccattcaagagatggatgtttgaagccaagctttttt

SEQ ID NO: 12 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 13 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 451 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWSFNPPTFGQGTKVEIKR

SEQ ID NO: 17 - protein, 122 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSASYWYFDVWGQGTLVTVSS

SEQ ID NO: 18 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWAFNPPTFGQGTKVEIKR

SEQ ID NO: 22 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 23 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 26 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 32 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIELTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 34 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 213 aa, Artificial sequence; identical to SEQ ID NO: 13
  sequence is synthesized

SEQ ID NO: 36 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 63 bp, Cricetulus griseus
agcttttccaaaaaagtgagacatgcacagacagtctcttgaactgtctgtgcatgtctcacg

SEQ ID NO: 38 - DNA, 63 bp, Cricetulus griseus
gattcgcttggcttcaaacatccattcaagagatggatgtttgaagccaagcttttttggaaa

SEQ ID NO: 39 - DNA, 63 bp, Cricetulus griseus
agcttttccaaaaaagcttggcttcaaacatccatctcttgaatggatgtttgaagccaagcg

SEQ ID NO: 40 - DNA, 64 bp, Cricetulus griseus
gatccgcctggagatatcattggtgttcaagagacaccaatgatatctccaggttttttggaaa

SEQ ID NO: 41 - DNA, 64 bp, Cricetulus griseus
agcttttccaaaaaacctggagatatcattggtgtctcttgaacaccaatgatatctccaggcg